SEQUENCE LISTING (24 sequences)

SEQ ID NO: 1 - DNA, 21 bp, Homo sapiens
ggctttgtgtgaaggcagaga

SEQ ID NO: 2 - DNA, 20 bp, Homo sapiens
tcgccagcatgaacagtgat

SEQ ID NO: 3 - DNA, 21 bp, Homo sapiens
tgccaattatctcccgaatcg

SEQ ID NO: 4 - DNA, 21 bp, Homo sapiens
aacagaccaacgtttcggacc

SEQ ID NO: 5 - DNA, 20 bp, Homo sapiens
acggtaaaccccaattacgc

SEQ ID NO: 6 - DNA, 22 bp, Homo sapiens
tgtcaggtagcggttgtagtga

SEQ ID NO: 7 - DNA, 22 bp, Homo sapiens
gtgtgcgaacaggacatctacg

SEQ ID NO: 8 - DNA, 21 bp, Homo sapiens
agacggcgtcttcagtgaaca

SEQ ID NO: 9 - DNA, 18 bp, Homo sapiens
gagccctgcaccgtcacc

SEQ ID NO: 10 - DNA, 21 bp, Homo sapiens
ttgatgtcctgggactcctcc

SEQ ID NO: 11 - DNA, 20 bp, Homo sapiens
cgatccacacggagtacttg

SEQ ID NO: 12 - DNA, 21 bp, Homo sapiens
ggatgcagaaggagatcactg

SEQ ID NO: 13 - DNA, 22 bp, Homo sapiens
gttcaccaccaacaatcgagtg

SEQ ID NO: 14 - DNA, 21 bp, Homo sapiens
gatatacttcatggccaggcg

SEQ ID NO: 15 - DNA, 21 bp, Homo sapiens
gccaattatctcccgaatcga

SEQ ID NO: 16 - DNA, 21 bp, Homo sapiens
ttcgtccaggcagtagctttg

SEQ ID NO: 17 - DNA, 21 bp, Homo sapiens
acggtaaaccccaattacgcc

SEQ ID NO: 18 - DNA, 19 bp, Homo sapiens
ttttccacttcatgcgccg

SEQ ID NO: 19 - DNA, 22 bp, Homo sapiens
cccttcagaggaaccagtggat

SEQ ID NO: 20 - DNA, 22 bp, Homo sapiens
ctttcacccgcattgtcatctc

SEQ ID NO: 21 - DNA, 19 bp, Homo sapiens
gatggcgtactgtgggccc

SEQ ID NO: 22 - DNA, 21 bp, Homo sapiens
ttgatgtcctgggactcctcc

SEQ ID NO: 23 - DNA, 20 bp, Homo sapiens
cgatccacacggagtacttg

SEQ ID NO: 24 - DNA, 21 bp, Homo sapiens
ggatgcagaaggagatcactg

(19) United States
(12) Patent Application Publication
(10) Pub. No.: US 2014/0154219 A1
(43) Pub. Date: Jun. 5, 2014

(54) METHODS AND COMPOSITIONS FOR LARGE-SCALE ISOLATION OF VERY SMALL EMBRYONIC-LIKE (VSEL) STEM CELLS

(71) Applicants: Janina Ratajczak, Louisville, KY (US); Mariusz Z. Ratajczak, Louisville, KY (US)

(72) Inventors: Janina Ratajczak, Louisville, KY (US); Mariusz Z. Ratajczak, Louisville, KY (US)

(73) Assignee: University of Louisville Research Foundation, Inc., Louisville, KY (US)

(21) Appl. No.: 14/008,796

(22) PCT Filed: Apr. 2, 2012
(86) PCT No.: PCT/US12/31869

§ 371 Date: Feb. 18, 2014

Related U.S. Application Data
(60) Provisional application No. 61/470,701, filed on Apr. 1, 2011.

(51) Int. Cl. A61K 35/12

(52) U.S. Cl. CPC A61K 35/12
USPC 424/93.7; 435/325; 435/373

(57) ABSTRACT

Methods for purifying very small embryonic-like (VSEL) stem cells from populations of cells suspected of including VSEL stem cells are provided. In some embodiments, the methods include (a) providing a population of cells suspected of including a VSEL stem cell; and (b) isolating a CD45^{neg}/GlyA^{neg}/CD133⁺/ALDH^{high} subpopulation, a CD45^{neg}/GiyA^{neg}/CD133⁺/ALD^{low} subpopulation, a CD45^{neg}/Lin^{neg}/SSEA-4⁺/ALDH^{high} subpopulation, a CD45^{neg}/Lin^{neg}/SSEA-4/AiO^{low} subpopulation, or any combination thereof from the population, whereby a VSEL stem cell is purified from the population. Also provided are methods for generating in vitro hematopoietic colonies derived from VSEL stem cells and methods for generating lympho-hematopoietic chimerism in a subject.

### CROSS REFERENCE TO RELATED APPLICATIONS

This application is based on and claims the benefit of U.S. Provisional Application Ser. No. 61/470,701, filed Apr. 1, 2011, the disclosure of which is herein incorporated by reference in its entirety.

### GOVERNMENT INTEREST

This invention was made with U.S. Government support under Grant Nos. R01 CA106281-01 and R01 DK074720 awarded by U.S. National Institutes of Health. The U.S. Government has certain rights in the invention.

### TECHNICAL FIELD

The presently disclosed subject matter relates, in general, to the methods and compositions for isolating a population of stem cells that are referred to herein as very small embryonic-like (VSEL) stem cells from bone marrow, umbilical cord blood, and/or other sources.

### BACKGROUND

The use of stem cells and stem cell derivatives has gained increased interest in medical research, particularly in the area of providing reagents for treating tissue damage either as a result of genetic defects, injuries, and/or disease processes. Ideally, cells that are capable of differentiating into the affected cell types could be transplanted into a subject in need thereof, where they would interact with the organ microenvironment and supply the necessary cell types to repair the injury.

For example, hematopoietic stem cells (HSCs) can be purified from bone marrow (BM), umbilical cord blood (UCB), and mobilized peripheral blood (mPB) by employing separation schemes based on the following: (i) the presence of exemplary stem cell antigens (e.g., CD34 and CD133); (ii) the absence of lineage differentiation markers (e.g., lin^{neg}); (iii) high expression of aldehyde dehydrogenase (ALDH); and (iv) low accumulation of the dyes Hoechst 3342 (Hoe3342^{low}), Pyronin Y (Pyronin Y^{low}), and/or Rhodamine 123 (Rh123^{low}). See Ratajczak, 2008. A combination of signaling lymphocyte activating molecules (SLAM) markers (e.g., CD150⁺, CD48^{neg}, and CD244^{neg}) has also been recently proposed for discriminating primitive hematopoietic steal/progenitor cell (HSPC) populations (Kiel et al., 2005).

Nevertheless, the phenotype of most primitive hematopoietic stem cells (HSCs), known as long-term repopulating HSCs (LT-HSCs), is not very well defined. According to their definition, LT-HSCs establish long-lasting, stable chimerism after hematopoietic transplantation and, as proposed, reside in human BM among CD34^{neg}/CD38^{neg}/lin^{neg} (Gallacher et al., 2000) or CD133⁺/Lin^{neg}/ALDH^{high} cells (Hess et al., 2006). These most primitive HSCs display only limited clonogenic potential in routine assays in vitro; however, they are able to engraft and establish hematopoiesis in experimental animals (Larochelle et al., 1996; Bhatia et al., 1998). Similar cells can be detected in UCB by employing direct intra-bone marrow transplantation and have been identified among CD34^{neg}/flt^{neg}/lin^{neg} cells (Wang et al., 2003).

Recently, a population of very small embryonic/epiblast-like (VSEL) stem cells that (i) are smaller than erythrocytes; (ii) are SSEA-1⁺/Oct-4⁺/Sca-1⁺/CXCR4⁺/Lin^{neg}/CD45^{neg}; (iii) respond to an SDF-1 gradient; and (iv) have high nuclear:cytoplasm ratio and primitive euchromatin were identified in murine BM and fetal liver (FL). See Kucia et al., 2006b. Murine VSELs do not reveal hematopoietic activity immediately after isolation, but acquire hematopoietic potential similar to stem cells from established embryonic stem (ES) cell lines and induced pluripotent stem (iPS) cells following co-culture/activation over OP9 stroma (Ratajczak et al., 2011). Based on these findings, it was hypothesized that in murine BM they fulfill the functional criteria for LT-HSCs (Ratajczak et al., 2011).

As recently demonstrated, these cells are also early in the mesenchymal lineage hierarchy (Taichman et al., 2010). Corresponding populations of SSEA-4⁺/Oct-4⁺/CD133⁺/CD34⁺/CXCR4⁺/Lin^{neg}/CD45^{neg} cells in UCB that are (i) slightly smaller than erythrocytes; (ii) have high nuclear:cytoplasm ratio and euchromatin; and (iii) display transcription factor signature of pluripotent stem cells (PSCs), including Oct-4 and Nanog, have also been identified (Kucia et al., 2007). By employing fluorescence-activated cell sorting (FACS) followed by quantitative genetic analysis, it was determined that CD45^{neg}/Lin^{neg}/CD133⁺, CD45^{neg}/Lin^{neg}/CD34⁺, and CD45^{neg}/Lin^{neg}/CXCR4⁺ fractions of UCB cells are significantly enriched in VSELs (tuba-Surma et al., 2010). However, molecular analysis revealed that the subpopulation of CD45^{neg}/Lin^{neg}/CD133⁺ cells, a rare CD45^{neg}/Lin^{neg} cell population, possesses the highest expression of pluripotency markers. Based on this, it was hypothesized that CD133 antigen might be a useful surface marker to identify the most primitive VSELs. The presence of similar cells was recently confirmed in UCB, human BM, and mPB (McGuckin et al., 2008; Sovalat et al., 2011).

### SUMMARY

This Summary lists several embodiments of the presently disclosed subject matter, and in many cases lists variations and permutations of these embodiments. This Summary is merely exemplary of the numerous and varied embodiments. Mention of one or more representative features of a is given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides methods for purifying very small embryonic-like (VSEL) stem cells from populations of cells suspected of comprising VSEL stem cells. In some embodiments, the methods comprise (a) providing a population of cells suspected of comprising a VSEL stem cell; and (b) isolating a CD45^{neg}/GlyA^{neg}/CD133⁺/ALDH^{high} subpopulation, a CD45^{neg}/GlyA^{neg}/CD133⁺/ALDH^{low} subpopulation, a CD45^{neg}/Lin^{neg}/SSEA-4⁺/ALDH^{high} subpopulation, a CD45^{neg}/Lin^{neg}/SSEA-4/ALDH^{low} subpopulation, or any combination thereof from the population, whereby a VSEL stem cell is purified from the population. In some embodiments, the isolating step comprises employing anti-CD133 paramagnetic beads to isolate a CD133⁺ subpopulation from the population. In some embodiments, the isolating step comprises employing an anti-SSEA-4 antibody to isolate an SSEA-4⁺ subpopulation from the population. In some embodiments, the isolating step comprises employing a fluorescent dye to detect aldehyde dehydrogenase (ALDH) expression in the cells of the population, the CD133⁺ subpopulation from the population, the SSEA-4⁺ subpopulation from the population, or in any other subpopulation thereof. In some embodiments, the fluorescent dye is employed for separating the cells into ALDH^{high} and ALDH^{low} fractions. In some embodiments, the isolating comprises employing a reagent that binds to Glycophorin A (GlyA) to remove GlyA⁺ cells from the population. In some embodiments, the isolating comprises employing a reagent that binds to CD45 to remove CD45⁺ cells from the population. In some embodiments, the population of cells suspected of comprising VSEL stem cells is a bone marrow sample, a peripheral blood sample, a spleen sample, an umbilical cord blood sample, or any combination thereof.

The presently disclosed subject matter also provides in some embodiments methods for generating in vitro hematopoietic colonies derived from very small embryonic-like (VSEL) stem cells. In some embodiments, the methods comprise (a) providing a CD45^{neg}/GlyA^{neg}/CD133⁺/ALDH^{high} subpopulation, a CD45^{neg}/GlyA^{neg}/CD133⁺/ALDH^{low} subpopulation, a CD45^{neg}/Lin^{neg}/SSEA-4⁺/ALDH^{high} subpopulation, a CD45^{neg}/Lin^{neg}/SSEA-4/ALDH^{low} subpopulation, or any combination thereof purified by a method of the presently disclosed subject matter; and (b) co-culturing the VSEL stem cell present therein in the presence of an OP9 stromal cell feeder layer under conditions sufficient to generate an in vitro hematopoietic colony derived from the VSEL stem cell. In some embodiments, the conditions sufficient to generate an in vitro hematopoietic colony derived from the VSEL stem cell comprise co-culturing the VSEL stem cell in the presence of the OP9 stromal cell feeder layer for at least 5 days, optionally for at least 7 days, and further optionally for at least 10 days.

In some embodiments, the presently disclosed subject matter also provides methods for generating lympho-hematopoietic chimerism in a subject. In some embodiments, the methods comprise introducing into a lympho-hematopoietic compartment of the subject a plurality of CD45^{neg}/GlyA^{neg}/CD133⁺/ALDH^{high} cells comprising VSEL stem cells, a plurality of CD45^{neg}/GlyA^{neg}/CD133⁺/ALDH^{low} cells comprising VSEL stem cells, a plurality of CD45^{neg}/Lin^{neg}/SSEA-4⁺/ALDH^{high} cells comprising VSEL stem cells, a plurality of CD45^{neg}/Lin^{neg}/SSEA-4⁺/ALDH^{low} cells comprising VSEL stem cells, or a combination thereof, wherein the plurality of CD45^{neg}/GlyA^{neg}/CD133⁺/ALDH^{high} cells comprising VSEL stem cells, the plurality of CD45^{neg}/GlyA^{neg}/CD133⁺/ALDH^{low} cells comprising VSEL stem cells, the plurality of CD45^{neg}/Lin^{neg}/SSEA-4⁺/ALDH^{high} cells comprising VSEL stem cells, the plurality of CD45^{neg}/Lin^{neg}/SSEA-4⁺/ALDH^{low} cells comprising VSEL stem cells, or the combination thereof were cultured over an OP9 stromal cell feeder layer under conditions sufficient to induce lympho-hematopoietic competency in one or more VSEL stem cells present therein. In some embodiments, the introducing comprises administering the plurality of CD45^{neg}/GlyA^{neg}/CD133⁺/ALDH^{high} cells comprising VSEL stem cells, the plurality of CD45^{neg}/GlyA^{neg}/CD133⁺/ALDH^{low} cells comprising VSEL stem cells, the plurality of CD45^{neg}/Lin^{neg}/SSEA-4⁺/ALDH^{high} cells comprising VSEL stem cells, the plurality of CD45^{neg}/Lin^{neg}/SSEA-4⁺/ALDH^{low} cells comprising VSEL stem cells, or the combination thereof to the subject intravenously. In some embodiments, the introducing step comprises a sufficient number of VSEL stem cells to repopulate bone marrow of the subject with lympho-hematopoietic cells derived from the VSEL stem cells. In some embodiments, the subject is a mammal, optionally a human.

Thus, it is an object of the presently disclosed subject matter to provide methods and compositions for isolating populations comprising VSEL stem cells from various sources.

An object of the presently disclosed subject matter having been stated hereinabove, and which is achieved in whole or in part by the presently disclosed subject matter, other objects will become evident as the description proceeds when taken in connection with the accompanying drawings as best described herein below.

### BRIEF DESCRIPTION OF THE DRAWINGS

FIG. 1 depicts exemplary three-step strategies for larger-scale preparation of VSELs from UCB. The three-step isolation strategies are depicted based on removal of red blood cells (RBCs) by hypotonic lysis (Step 1) followed by immunomagnetic separation of CD133⁺ cells (Step 2), followed by FACS-based isolation of either (a) CD133⁺/Lin^{neg}/CD45^{neg} and CD133⁺/Lin^{neg}/CD45⁺ subpopulations (Step 3, option a; left); or (b) CD133⁺/GlyA⁺/CD45^{neg} or CD133⁺/GlyA⁺/CD45⁺ cells that are ALDH^{high} or ALDH^{low} by combining exposure of the CD133⁺ cells to an ALDEFLUOR® reagent (i.e., a non-immunological aldehyde dehydrogenase (ALDH) detection reagent available from STEMCELL™ Technologies, Vancouver, British Columbia, Canada) prior to AFCS sorting (Step 3, option b; right). Employing this second strategy, it is possible to isolate VSELs (CD45^{neg}/GlyA^{neg}/CD133⁺/ALDH^{high} and CD45^{neg}/GlyA^{neg}/CD133⁺/ALDH^{low}), as well as hematopoietic stem/progenitor cells (HSPCs; CD45⁺/GlyA^{neg}/CD133⁺/ALDH^{high} and CD45⁺/GlyA^{neg}/CD133⁺/ALDH^{low}) as four separate subpopulations.

FIGS. 2A-2D depict exemplary gating strategies for FACS sorting of UCB VSELs and HSCs based on expression of particular markers, and the results thereof.

FIG. 2A is a series of FACS scatter plots depicting exemplary gating strategies for FACS sorting of UCB VSELs and HSCs based on expression of CD133 and CD45, and ALDH activity. UCB nucleated cell populations were stained using monoclonal antibodies against human CD235a (GlyA), CD45, and CD133 and exposed to an ALDEFLUOR® ALDH detection reagent. Sort gates were established by sequentially gating on FSC vs. SSC in region R1, followed by gating to define CD45^{neg}/GlyA^{neg} (region R2) and CD45^{neg}/GlyA^{neg} (region R3) populations. Cell populations were also defined based on their ALDH activity. Accordingly, cells were sorted as CD45^{neg}/GlyA^{neg}/CD133⁺/ALDH^{low} (region R4) and CD45^{neg}/GlyA^{neg}/CD133⁺/ALDH^{high} (region R5) subpopulations of VSELs, and as CD45⁺/GlyA^{neg}/CD133⁺/ALDH^{low} (region R6) and CD45⁺/GlyA^{neg}/CD133⁺1 ALDH^{high} (region R7) hematopoietic stem cell (HSC) populations.

FIG. 2B is a bar graph showing the percentage of all fractions of sorted VSELs (CD45^{neg}/ALDH^{low} and CD45^{neg}/ALDH^{high}) and HSPCs (CD45⁺/ALDH^{low} and CD45⁺/ALDH^{high}) among UCB-derived CD133⁺/GlyA^{neg} cells. The data shown represent the combined results from six independent experiments.

FIG. 2C is a series of FACS scatter plots depicting exemplary gating strategies for FACS sorting of UCB VSELs and HSCs based on expression of CD133, CD45, and lineage markers. UCB-VSELs were isolated from fraction of human UCB total nucleated cells (TNCs) by FACS by employing following gating criteria. In FIG. 2C, panel 1, all events ranging from 2 μm were included in gate R1 after comparison with six differently sized bead particles with standard diameters of 1, 2, 4, 6, 10 and 15 μm. In FIG. 2C, panel 2, UCB-derived TNCs were visualized on a dot plot based on FSC vs. SSC signals. In FIG. 2C, panel 3, cells from region R1 were further analyzed for CD133 and Lin expression: Lin^{neg}/CD133⁺ events were included in region R2. In FIG. 2C, panel 4, the Lin^{neg}/CD133⁺ population from region R2 was subsequently analyzed based on CD45 antigen expression and CD45^{neg} and CD45⁺ subpopulations visualized on dot plot; i.e., CD133⁺/Lin^{neg}/CD45^{neg} (VSELs: region R3) and CD133⁺/Lin^{neg}/CD45⁺ (HSCs: region R4).

FIG. 2D is a series of FACS scatter plots depicted exemplary gating strategies for FACS sorting of UCB VSELs and HSCs based on expression of SSEA-4, CD45, and lineage markers. SSEA-4⁺/Lin^{neg}/CD45^{neg} cells were isolated from fraction of human UCB TNCs by FACS by employing following gating criteria. In FIG. 2D, panel 1, all events ranging from 2 μm were included in gate R1 after comparison with six differently sized bead particles with standard diameters of 1, 2, 4, 6, 10 and 15 μm. In FIG. 2D, panel 2, UCB-derived TNCs were visualized on a dot plot based on FSC vs. SSC signals. In FIG. 2D, panel 3, cells from region R1 were further analyzed for SSEA-4 and Lin expression: Lin^{neg}/SSEA-4⁺ events were included in region R2. In FIG. 2D, panel 4, the Lin^{neg}/SSEA-4⁺ population from region R2 was subsequently analyzed based on CD45 antigen expression and CD45^{neg} and CD45⁺ subpopulations visualized on dot plot; i.e., SSEA-4⁺/Lin^{neg}/CD45^{neg} (region R3) and SSEA-4⁺/Lin^{neg}/CD45⁺ (region R4).

FIGS. 3A-3C show the results of various experiments testing hematopoietic differentiation of VSELs.

FIG. 3A depicts the results of fluorescence immunohistochemistry of UCB-derived CD45^{neg}/GlyA^{neg}/CD133⁺/ALDH^{low} cells with antibodies directed against Oct-4, Nanog, and SSEA-4. As shown in these panels, these cells expressed SSEA-4, Oct-4, and Nanog. All images were taken under a Plan Apo 60XA/1.40 oil objective (Nikon, Japan). Nuclei were visualized after DAR staining. Staining was performed on cells isolated from four independent sortings. Representative data are shown, FIG. 3B is a bar graph showing the results of reverse transcription-polymerase chain reaction (RT-PCR) analyses of the expression of hematopoietic genes in freshly isolated VSELs and HSCs. The data shown represent the combined results from four independent experiments carried out in triplicate per group (n=12). The first bar in each group of four (light grey) represents expression of the noted gene product in CD45⁺/GlyA^{neg}/CD133⁺/ALDH^{high} cells, the second bar in each group of four (white) represents expression of the noted gene product in CD45⁺/GlyA^{neg}/CD133⁺/ALDH^{low} cells, the third bar in each group of four (dark gray) represents expression of the noted gene product in CD45^{neg}/GlyA^{neg}/CD133⁺/ALDH^{high} cells, and the fourth bar in each group of four (black) represents expression of the noted gene product in CD45^{neg}/GlyA^{neg}/CD133⁺/ALDH^{low} cells. FIG. 3C depicts a representative gel of the RT-PCR analyses described herein above with respect to FIG. 3B.

FIGS. 4A-4D depict the results of experiments that demonstrated that VSELs were specified into HSCs in co-cultures over OP9 stromal cells.

FIG. 4A is a bar graph showing that in contrast to CD45⁺/GlyA^{neg}/CD133⁺/ALDH^{high} HSPCs, VSELs freshly isolated from murine BM did not grow hematopoietic colonies. FIG. 4B depicts photomicrographs of UCB-derived CD45^{neg}/GlyA^{neg}/CD133⁺/ALDH^{low} (top panel) and CD45^{neg}/GlyA^{neg}/CD133⁺/ALDH^{high} VSELs (bottom panel) grown over OP9 stromal cells at day 7. Representative pictures are shown at 20× magnification. FIG. 4C is two bar graphs showing the number of colonies formed in methylcellulose by OP9-primed VSELs and HSCs (left panel), as well as VSEL-derived and HSC-derived cells replated after 10 days in secondary methylcellulose cultures (right panel). The data shown in FIGS. 4A and 4C represent the combined results from four independent experiments carried out in triplicate for each group (n=12). *p<0.05; **p<0.0001. FIG. 4D is a bar graph showing the results of FACS analyses of hematopoietic gene expression on cells isolated from colonies formed in methylcellulose by OP9-cultured UCB-derived CD45^{neg}/GlyA^{neg}/CD133⁺/ALDH^{high} (black bars) and CD45^{neg}/GlyA^{neg}/CD133⁺/ALDH^{low} (white bars) VSELs. The data shown represent the combined results from three independent experiments carried out in duplicate per group (n=6).

FIGS. 5A-5C are bar graphs showing donor-derived cell populations present in bone marrow (BM; FIG. 5A), spleen (FIG. 5B), and peripheral blood (FIG. 5C) in mice after in vivo transplantation of freshly sorted UCB-derived VSELs and HSPCs with 10⁶ CD45⁺ OP9-cultured cells. FIGS. 5A-5C show the results of analyses of cells expressing human CD45, CD3, CD19, CD66b, and GlyA performed 6 weeks after transplantation with OP9-cultured CD45^{neg}/GlyA^{neg}/CD133⁺/ALDH^{low} cells (white boxes), OP9-cultured CD45^{neg}/GlyA^{neg}/CD133⁺/ALDH^{high} cells (black boxes), OP9-cultured CD45⁺/GlyA^{neg}/CD133⁺/ALDH^{low} cells (hatched boxes), or OP9-cultured CD45⁺/GlyA^{neg}/CD133⁺/ALDH^{high} cells (gray boxes). The data shown represent the combined results from 6 mice per group (n=6).

FIG. 6 is a bar graph showing a comparison of hypotonic lysis (dark gray bars) vs. FICOLL-PAQUE™ (light gray bars) removal of erythrocytes from populations of cells comprising VSELS. UCB samples were divided in half and erythrocytes were removed either by hypotonic lysis (dark gray bars) or FICOLL-PAQUE™ centrifugation (light gray bars). The left panel shows the number of CD34⁺ and CD133⁺/Lin^{neg}/CD45^{neg} VSELs. The right panel shows the number of CD34⁺ and CD133⁺/Lin^{neg}/CD45⁺ HSPCs.

### BRIEF DESCRIPTION OF THE SEQUENCE LISTING

SEQ ID NOs: 1-24 are the nucleotide sequences of 32 primer pairs that can be used to amplify nucleic acid sequences from various genetic loci (e.g., human genetic loci) as summarized in Tables I and II below.

### DETAILED DESCRIPTION

### I. General Considerations

Disclosed herein are methods for isolating and/or purifying VSELs from sources suspected of containing VSELs, and compositions comprising VSELs so isolated. In an exemplary embodiment, anti-CD133 paramagnetic beads, followed by staining with ALDEFLUORC® (a reagent that can be used to detect and/or assay aldehyde dehydrogenase (ALDH) activity), and sorting CD45^{neg}/GlyA^{neg}/CD133⁺/ALDH^{high} and CD45^{neg}/GlyA^{neg}/CD133⁺/ALDH^{low} subpopulations of VSELs and CD45⁺/GlyA^{neg}/CD133⁺/ALDH^{high} and CD45⁺/GlyA^{neg}/CD133⁺/ALDH^{low} subpopulations of hematopoietic stem/progenitor cells (HSPCs) was employed to attain a faster process to avoid time-consuming multiparameter sorting procedure for isolating and/or purifying VSELs from UCB. It was determined that while freshly isolated VSELs did not grow hematopoietic colonies in vitro, these cells, if immediately expanded over OP9 stromal cells, acquired hematopoietic potential and grew colonies comprising CD45⁺ cells.

Furthermore, while CD45⁺ cells gave raise to hematopoietic colonies after the first replating, the formation of colonies by CD45^{neg}/GlyA^{neg}/CD133⁺/ALDH^{low} VSELs was somewhat delayed, suggesting that these cells might require more time to attain hematopoietic commitment. In parallel, real-time PCR analysis confirmed that while freshly isolated CD45^{neg}/GlyA^{neg}/CD133⁺/ALDH^{high} VSELs express more hematopoietic transcripts, CD45^{neg}/GlyA^{neg}/CD133⁺/ALDH^{low} VSELs exhibit higher levels of pluripotent stem cell transcription factors.

Finally, by employing in vivo transplants into NOD/SCID mice, it was determined that both CD45^{neg}/GlyA^{neg}/CD133⁺/ALDH^{high} and CD45^{neg}/GlyA^{neg}/CD133⁺/ALDH^{low} VSELs cultured over OP9 cells can give rise to human lympho-hematopoietic chimerism as assayed 4-6 weeks after transplantation. Although it is not desired that the presently disclosed subject matter be limited to any particular theory of operation, it is possible that like murine BM-derived VSELs, human UCB-derived CD45^{neg} VSELs correspond to a population of the most primitive long-term repopulating HSCs (LT-HSCs).

### II. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. For example, the phrase “a cell” refers to one or more cells, including, but not limited to a plurality of the same cell type or a plurality of different cell types. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, and in some embodiments ±0.01% from the specified amount, as such variations are appropriate to perform the disclosed methods. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. For example, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the is disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent present in the pharmaceutical composition. It is noted, however, that carriers, excipients, and other inactive agents can and likely would be present in the pharmaceutical composition.

With respect to the terms “comprising”, “consisting essentially of”, and “consisting of”, where one of these three terms is used herein, the presently disclosed and claimed subject matter can include the use of either of the other two terms. For example, the presently disclosed subject matter relates in some embodiments to compositions that comprise CD133⁺/GlyA^{neg}/CD45^{neg} cells. It is understood that the presently disclosed subject matter thus also encompasses compositions that in some embodiments consist essentially of CD133⁺/GlyA^{neg}/CD45^{neg} cells, as well as compositions that in some embodiments consist of CD133⁺/GlyA^{neg}/CD45^{neg} cells. Similarly, it is also understood that in some embodiments the methods of the presently disclosed subject matter comprise the steps the steps that are disclosed herein and/or that are recited in the claims, in some embodiments the methods of the presently disclosed subject matter consist essentially of the steps that are disclosed herein and/or that are recited in the claims, and in some embodiments the methods of the presently disclosed subject matter consist of the steps that are disclosed herein and/or that are recited in the claim.

As used herein, the phrase “long term” when used in the context of bone marrow transplantation refers to a period of time in which the donor cell or a progeny cell derived therefrom remains viable and functional in the donor. Bone marrow transplantation is considered to result in long term engraftment when hematopoietic cells derived from the donor cells are present in the recipient for in some embodiments at least 3 months, in some embodiments 6 months, in some embodiments 9 months, in some embodiments 12 months, and in some embodiments for longer than 12 months after administration.

### III. Methods for Purifying VSEL Stem Cells and Subpopulations of Purified VSEL Stem Cells

The presently disclosed subject matter provides in some embodiments methods of isolating and/or purifying VSEL stem cells, optionally from populations of cells that are suspected of comprising VSEL stem cells. In some embodiments, the methods comprise (a) providing a population of cells suspected of comprising a VSEL stem cell; and (b) isolating a CD45^{neg}/GlyA^{neg}/CD133⁺/ALDH^{high} subpopulation, a CD45^{neg}/GlyA^{neg}/CD133⁺/ALDH^{low} subpopulation, or both from the population, whereby a VSEL stem cell is purified from the population.

As used herein, the term “CD45” refers to a tyrosine phosphatase, also known as the leukocyte common antigen (LCA), and having the gene symbol PTPRC. This gene corresponds to GENBANK® Accession Nos. NP_{—}002829 (human), NP_{—}035340 (mouse), NP_{—}612516 (rat), XP_{—}002829 (dog), XP_{—}599431 (cow) and AAR16420 (pig). The amino acid sequences of additional CD45 homologs are also present in the GENBANK® database, including those from several fish species and several non-human primates. In some embodiments, a subpopulation of CD45^{neg} cells is isolated from a mixed population of CD45′ and CD45^{neg} cells. In some embodiments, the CD45^{neg} subpopulation is prepared by employing a reagent that binds to CD45 to remove CD45⁺ cells from a mixed population comprising both CD45⁺ and CD45^{neg} cells.

As used herein, the term “CD34” refers to a cell surface marker found on certain hematopoietic and non-hematopoietic stem cells, and having the gene symbol CD34. The GENBANK® database discloses amino acid and nucleic acid sequences of CD34 from humans (e.g., AAB25223), mice (NP_{—}598415), rats (XP_{—}223083), cats (NP_{—}001009318), pigs (MP_{—}999251), cows (NP_{—}776434), and others. In some embodiments, a population of cells is separated into two subpopulations, with one subpopulations consisting essentially of CD34⁺ cells and the other subpopulation consisting essentially of CD34^{neg} cells.

In mice, some stem cells also express the stem cell antigen Sca-1 (GENBANK® Accession No, NP_{—}034868), which is also referred to as Lymphocyte antigen Ly-6A.2.

As used herein, the term “CD133” refers to a cell surface marker found on certain hematopoietic stem cells, endothelial progenitor cells, glioblastomas, neuronal and glial stem cells, and some other cell types. It is also referred to as Prominin 1 (PROM1). The GENBANK® database discloses nucleic acid and amino acid sequences of CD133 from humans (e.g., NM_{—}006017 and NP_{—}006008), mice (NM_{—}008935 and NP_{—}032961), rats (NM_{—}021751 and NP_{—}068519), and others. In some embodiments, a subpopulation of CD133⁺ cells is isolated from a mixed population of CD133⁺ and CD133^{neg} cells. In some embodiments, the CD133⁺ subpopulation is prepared by employing a reagent that binds to CD133 to isolate CD133⁺ cells from a mixed population that comprises both CD133⁺ and CD133^{neg} cells.

As used herein, the term “GlyA” refers to glycophorin A, a cell surface molecule present on red blood cells. The GENBANK® database discloses nucleic acid and amino acid sequences of GlyA from humans (e.g., NM_{—}002099 and NP_{—}002090), mice (NM_{—}010369 and NP_{—}034499), and others. In some embodiments, a subpopulation of GlyA^{neg} cells is isolated from a mixed population of GlyA⁺ and GlyA^{neg} cells. In some embodiments, the GlyA^{neg} subpopulation is prepared by employing a reagent that binds to GlyA to remove GlyA⁺ cells from a mixed population that comprises both GlyA⁺ and GlyA^{neg} cells.

Thus, the subpopulation of CD45^{neg} stem cells represents in some embodiments a subpopulation of CD45^{neg} cells that are present in the population of cells prior to the separating step. In some embodiments, the subpopulation of CD45^{neg} stem cells is from a human, and is CD34⁺/lin^{neg}/CD45^{neg}. In some embodiments, the subpopulation of CD45^{neg} stem cells is from a mouse, and is Sca-1⁺/lin^{neg}/CD45^{neg}. In some embodiments, the subpopulation of CD45^{neg} stem cells is also GlyA^{neg}.

The isolation of the disclosed subpopulations can be performed using any methodology that can separate cells based on expression or lack of expression of the one or more markers selected from among CD45, CD133, GlyA, CXCR4, CD34, AC133, Sca-1, CD45R/B220, Gr-1, TCRaβ, TCRγδ, CD11b, and Ter-119. In some embodiments, the methodology employs a technique including, but not limited to fluorescence-activated cell sorting (FACS).

As used herein, ling refers to a cell that does not express any of the following markers: CD45R/B220, Gr-1, TCRaβ, TCRγδ, CD11b, and Ter-119. These lineage markers are generally found on cells of the B cell lineage from early Pro-B to mature B cells (CD45R/B220); cells of the myeloid lineage such as monocytes during development in the bone marrow, bone marrow granulocytes, and peripheral neutrophils (Gr-1); thymocytes, peripheral T cells, and intestinal intraepithelial lymphocytes (TCRaβ and TCRγδ); myeloid cells, NK cells, some activated lymphocytes, macrophages, granulocytes, B1 cells, and a subset of dendritic cells (CD11 b); and mature erythrocytes and erythroid precursor cells (Ter-119).

The separation step can be performed in a stepwise or iterative manner (e.g., as a series of steps) or the one or more of the steps can occur concurrently. For example, the presence or absence of each marker can be assessed individually, producing two subpopulations at each step based on whether the individual marker is present. Thereafter, the subpopulation of interest can be selected and further divided based on the presence or absence of the next marker.

Alternatively, the subpopulation can be generated by separating out only those cells that have a particular marker profile, wherein the phrase “marker profile” refers to a summary of the presence or absence of two or more markers. For example, a mixed population of cells can contain both CD133⁺ and CD133^{neg} cells. Similarly, the same mixed population of cells can contain both CD45⁺ and CD45^{neg} cells. Thus, certain of these cells will be CD133⁺/CD45⁺, others will be CD133⁺/CD45^{neg}, others will be CD133^{neg}/CD45⁺, and others will be CD133^{neg}/CD45^{neg}. Each of these individual combinations of markers represents a different marker profile. As additional markers are selected for and/or against, the profiles can become more complex and correspond to a smaller and smaller percentage of the original mixed population of cells. In some embodiments, the cells of the presently disclosed subject matter have a marker profile of CD133⁺/CD45^{neg}/GlyA^{neg}.

In some embodiments of the presently disclosed subject matter, antibodies specific for markers expressed by a cell type of interest (e.g., polypeptides expressed on the surface of a CD133⁺/CD45^{neg}/GlyA^{neg} cell) are employed for isolation and/or purification of subpopulations of BM, UCB, spleen, and/or peripheral blood cells that have marker profiles of interest. It is understood that based on the marker profile of interest, the antibodies can be used to positively or negatively select fractions of a population, which in some embodiments are then further fractionated. In some embodiments, antibodies are used to positively select cells that express markers that are present on VSELs (e.g., CD133, SSEA-1 or -4, CXCR4, CD34, AP, c-met, and/or LIF-R), and subpopulations of cells that express these markers are retained and in some embodiments further purified. In some embodiments, antibodies are used to negatively select cells that express markers that are not present on VSELs (e.g., CD45, GlyA, lineage markers such as, but not limited to CD45R/B220, Gr-1, TCRaβ, TCRγδ, CD11b, and Ter-119), and cells that express these markers are removed to produce a subpopulation of cells that in some embodiments can be further purified.

In some embodiments, a plurality of antibodies, antibody derivatives, and/or antibody fragments with different specificities is employed. In some embodiments, each antibody, or fragment or derivative thereof that contains at least one antigen-binding domain (referred to herein as a “paratope”), is specific for a marker selected from the group including but not limited to CD133, CD45, GlyA, Ly-6A/E (Sca-1), CD34, CXCR4, AC133, CD45, CD45R, B220, Gr-1, TCRαβ, TCRγδ, CD11 b, Ter-119, c-met, LIF-R, SSEA-1 and/or SSEA-4, Oct-4, Rev-1, and Nanog. In some embodiments, cells that express one or more genes selected from the group including but not limited to CD133, SSEA-1, Oct-4, Rev-1, and/or Nanog are isolated and/or purified.

The presently disclosed subject matter relates to a population of cells that in some embodiments are CD133⁺/CD45^{neg}/GlyA^{neg}, and in some embodiments these cells also express the following antigens: CXCR4, AC133, CD34, SSEA-1 (mouse) or SSEA-4 (human), fetal alkaline phosphatase (AP), c-met, and the LIF-Receptor (LIF-R). In some embodiments, the cells of the presently disclosed subject matter do not express the following antigens: CD45, lineage markers (i.e., the cells are lin^{neg}), GlyA, HLA-DR, MHC class I, CD90, CD29, and CD105. Thus, in some embodiments the cells of the presently disclosed subject matter can be characterized as follows: CD133⁺; CD45^{neg}; GlyA^{neg}; CXCR4⁺; CD133⁺; CD34⁺; SSEA-1⁺ (mouse) or SSEA-4⁺ (human); AP⁺; c-met⁺; LIF-R⁺; HLA-DR^{neg}; MHC class I^{neg}; CD90^{neg}; CD29^{neg}; CD105^{neg}.

Alternatively or in addition, the presently disclosed subject matter provides methods of isolating and/or purifying VSEL stem cells, optionally from populations of cells that are suspected of comprising VSEL stem cells, that comprise (a) providing a population of cells suspected of comprising a VSEL stem cell; and (b) isolating an SSEA-4⁺/lin^{neg}/CD45^{neg} subpopulation, whereby a VSEL stem cell is purified from the population. Based on immunohistochemical staining data that UCB-VSELs highly express SSEA-4 on the cell surface, an antibody that binds to SSEA-4 can also be employed for FACS sorting. By employing this antibody, it is possible to purify UCB-VSELs as a population of SSEA-4⁺/Lin^{neg}/CD45^{neg} cells.

It is understood that in order to isolate a subpopulation of cells with the marker profile desired (e.g., CD133⁺/CD45^{neg}/GlyA^{neg} and/or SSEA-4⁺/Lin^{neg}/CD45^{neg}), the ligands that are used to separate cells based on expression of the relevant markers (e.g., antibodies) can be employed simultaneously or iteratively, in any combination that is convenient. For example, antibodies that bind to CD133, CD45, and GlyA and/or SSEA-4 can be employed simultaneously, in any desired combinations, or singly, in any order that might be convenient to separate the desired subpopulations.

In some embodiments, each antibody, or fragment or derivative thereof, comprises a detectable label. Different antibodies, or fragments or derivatives thereof, which bind to different markers can comprise different detectable labels or can employ the same detectable label.

A variety of detectable labels are known to the skilled artisan, as are methods for conjugating the detectable labels to biomolecules such as antibodies and fragments and/or derivatives thereof. As used herein, the phrase “detectable label” refers to any moiety that can be added to an antibody, or a fragment or derivative thereof, which allows for the detection of the antibody. Representative detectable moieties include, but are not limited to, covalently attached chromophores, fluorescent moieties, enzymes, antigens, groups with specific reactivity, chemiluminescent moieties, and electrochemically detectable moieties, etc. In some embodiments, the antibodies are biotinylated. In some embodiments, the biotinylated antibodies are detected using a secondary antibody that comprises an avidin or streptavidin group and is also conjugated to a fluorescent label including, but not limited to Cy3, Cy5, and Cy7. In some embodiments, the antibody, fragment, or derivative thereof is directly labeled with a fluorescent label such as Cy3, Cy5, or Cy7. In some embodiments, the antibodies comprise biotin-conjugated rat anti-mouse Ly-6A/E (Sca-1; clone E13-161.7), streptavidin-PE-Cy5 conjugate, anti-CD45-APCCy7 (clone 30-F11), anti-CD45R/E3220-PE (clone RA3-6B2), anti-Gr-1-PE (clone RB6-8C5), anti-TCRαβ PE (clone H57-597), anti-TCRγδ PE (clone GL3), anti-CD11 b PE (clone M1/70) and anti-Ter-119 PE (clone TER-119). In some embodiments, the antibody, fragment, or derivative thereof is directly labeled with a fluorescent label and cells that bind to the antibody are separated by fluorescence-activated cell sorting. Additional detection strategies are known to the skilled artisan.

While FACS scanning is a convenient method for purifying subpopulations of cells, it is understood that other methods can also be employed. An exemplary method that can be used is to employ antibodies that specifically bind to one or more of CD45, CXCR4, CD34, AC133, Sca-1. CD45R/B220, Gr-1, TCRaβ, TCRγδ, CD11 b, and Ter-119, with the antibodies comprising a moiety (e.g., biotin) for which a high affinity binding reagent is available (e.g., avidin or streptavidin). For example, a biotin moiety could be attached to antibodies for each marker for which the presence on the cell surface is desirable (e.g., CD34, Sca-1, CXCR4), and the cell population with bound antibodies could be contacted with an affinity reagent comprising an avidin or streptavidin moiety (e.g., a column comprising avidin or streptavidin). Those cells that bound to the column would be recovered and further fractionated as desired. Alternatively, the antibodies that bind to markers present on those cells in the population that are to be removed (e.g., CD45R/B220, Gr-1, TCRaβ, TCRγδ, CD11b, and Ter-119) can be labeled with biotin, and the cells that do not bind to the affinity reagent can be recovered and purified further.

It is also understood that different separation techniques (e.g., affinity purification and FACS) can be employed together at one or more steps of the purification process.

In some embodiments, a VSEL stem cell or derivative thereof also expresses a marker selected from the group including but not limited to c-met, c-kit, LIF-R, and combinations thereof. In some embodiments, the disclosed isolation methods further comprise isolating those cells that are c-met⁺, c-kit⁺, and/or LIF-R⁺.

In some embodiments, the VSEL stem cell or derivative thereof also expresses SSEA-1, Oct-4, Rev-1, and Nanog, and in some embodiments, the disclosed isolation methods further comprise isolating those cells that express these genes.

A number of techniques have been developed for fractionating heterogeneous mixtures of cells into various subpopulations of interest. These techniques are in some embodiments based on the size and density of the cells, specific binding properties that they possess, and their expression of surface antigens. The technique chosen can depend in some embodiments on the degree of purity required, the intended use of the selected cells, and/or the abundance of the cells of interest.

For example, density gradient centrifugation, velocity sedimentation, and counterflow centrifugal elutriation are techniques that can be employed to separate cells based on their physical properties such as size and density. While these techniques generally can suffice as pre-enrichment steps, they typically are neither accurate nor specific enough to yield pure populations of VSEL stem cells.

As set forth hereinabove, flow cytometry can be an extremely sensitive separation technique because it looks at each cell individually. It can distinguish multiple markers, their relative level of expression, the size and granularity of each cell, and can sort out specific cells into subpopulations of interest based largely on the availability of fluorescent reagents that bind to markers that distinguish between desirable and undesirable subpopulations.

Alternatively or in addition, the ability to immobilize an antibody on a solid phase has facilitated the processing of larger cell numbers in a relatively short time while still exploiting the specificity of the antigen/antibody interaction. Such antibody “panning” can be an effective technique for cell separation. An exemplary, non-limiting technique employs magnetic beads as a solid phase. Thus, in some embodiments, a population of cells suspected of comprising a VSEL stem cell is incubated with one or more antibodies that are bound to magnetic beads. The antibodies that are bound to magnetic beads can be antibodies that bind to a marker that is expressed by a VSEL (e.g., CD133⁺) and used to bind the cells of interest, and/or antibodies that bind to a marker that is not expressed by a VSEL (e.g., CD45, GlyA, or a lineage marker) and that can be used to remove cells that are not of interest. These antibodies can be employed together or iteratively, as the design of the separation method might warrant.

Also alternatively or in addition, the antibodies that are bound to the solid support (e.g., magnetic beads) can be secondary antibodies that bind to desired anti-marker antibodies. For example, if a separation strategy includes only the use of antibodies that bind to cells types of interest (e.g., VSELs), then the population of cells suspected of comprising VSEL stem cells can be incubated with an anti-marker antibody in a first step, and thereafter incubated with a magnetic bead coated with a secondary antibody that binds to the marker-specific antibody. By way of a more particular example and also not to be interpreted as a limitation, a step in the purification of CD133⁺/GlyA^{neg}/CD45^{neg} cells could include a first step of incubating a mouse anti-CD133 monoclonal antibody to a first population of cells suspected of comprising VSEL stem cells, and the CD133⁺ subpopulation of cells could be purified via a subsequent step that included further incubating the first population with a solid support (e.g., a magnetic bead) conjugated to sheep anti-mouse IgG secondary antibody. Recovery of the magnetic beads would thereafter be expected to simultaneously recover the CD133⁺ fraction of the first population, and elution of the CD133⁺ cells from the magnetic beads (in some embodiments by disrupting the binding of the anti-CD133 antibodies is and in some embodiments by disrupting the binding of the secondary antibodies) would be expected to result in the production of a subpopulation of CD133⁺ cells that could thereafter be further purified, as desired. In some embodiments, an isolating step of a purification method can comprise employing anti-CD133 paramagnetic beads to isolate a CD133⁺ subpopulation from the population.

A related method employs a biotin-labeled targeting ligand (e.g., an anti-marker antibody or a fragment thereof that includes a paratope that binds to the marker) such that a complex comprising the biotin-labeled targeting ligand bound to a cell expressing the appropriate marker can be purified based on the high affinity of biotin to avidin or streptavidin, which is provided on a support (e.g., a bead, a column, etc.). Biotin-labeled antibodies are commercially available from several suppliers (e.g., Miltenyi Biotec (CD133); BIOLEGEND® Inc. of San Diego, Calif., United States of America (CD45)). Alternatively, an anti-marker antibody can be biotinylated by any one of several methods, including but not limited to binding of biotin maleimide [3-(N-maleimidylpropionyl)biocytin] moiety to one or more cysteine residues of the anti-marker antibody (Tang & Casey, 1999), binding of biotin to a biotin acceptor domain, for example that described in K. pneumoniae oxaloacetate decarboxylase, in the presence of biotin ligase (Julien et al., 2000), attachment of biotin amine to reduced sulfhydryl groups (U.S. Pat. No. 5,168,037), and chemical introduction of a biotin group into a nucleic acid ligand (Carninci et al., 1996). In some embodiments, a biotin-labeled targeting ligand and the unlabeled same target ligand show substantially similar binding to a target molecule. As was described herein above with respect to magnetic beads, the use of biotin-labeled targeting ligands (e.g., antibodies) can be iterative or simultaneous, depending on the antibodies employed.

In some embodiments, a population of cells or a subpopulation thereof (e.g., a subpopulation of CD133⁺/GlyA^{neg}/CD45^{neg} cells of the presently disclosed subject matter) is further separated based on expression of aldehyde dehydrogenase (ALDH) in the cells of the population or the subpopulation. In some embodiments, an isolating step of the presently disclosed subject matter can comprise employing a fluorescent dye to detect ALDH expression in the cells of a population (e.g., a CD133⁺ subpopulation, a GlyA^{neg} subpopulation, a CD45^{neg} subpopulation, or any combination thereof). By way of example and not limitation, an ALDEFLUOR® ALDH detection reagent (STEMCELL Technologies, Vancouver, British Columbia, Canada) can be used to separate CD133⁺/GlyA^{neg}/CD45^{neg} cells based on ALDH staining. As such, the presently disclosed methods can in some embodiments further comprise isolating ALDH^{high} cells from the CD133⁺/GlyA^{neg}/CD45^{neg} cells, ALDH^{low} cells from the CD133⁺/GlyA^{neg}/CD45^{neg} cells, or both ALDH^{high} cells and ALDH^{low} cells separately from the CD133⁺/GlyA^{neg}/CD45^{neg} cells.

The populations of cells suspected of comprising VSEL stem cells can be any population of cells from which VSEL stem cells might be purified. By way of example and not limitation, the population of cells suspected of comprising VSEL stem cells is in some embodiments a bone marrow sample, in some embodiments a peripheral blood sample, in some embodiments a spleen sample, in some embodiments an umbilical cord blood sample, or in some embodiments any combination of the foregoing.

Additionally, the population of cells suspected of comprising VSEL stem cells can be from any animal from which VSEL stem cells might be desirably purified including, but not limited to mammals. Exemplary, non-limiting mammals are rodents (such as but not limited to rats and mice) and humans.

The presently disclosed subject matter also provides isolated subpopulations of VSEL stem cells (alternatively referred to herein as “purified subpopulations”, “subpopulations”, etc.), wherein the isolated subpopulations of stem cells comprises substantially purified CD133⁺/GlyA^{neg}/CD45^{neg} cells isolated from umbilical cord blood (hereinafter “UCB” or “CB”). The isolated subpopulations of stem cells can comprise CD133⁺/GlyA^{neg}/CD45^{neg}/ALDH^{high} cells, CD133⁺/GlyA^{neg}/CD45^{neg}/ALDH^{low} cells, or a combination thereof.

A population of cells containing the CD133⁺/CD45^{neg}/GlyA^{neg} cells of the presently disclosed subject matter can be isolated from any subject or from any source within a subject that contains them. In some embodiments, the population of cells comprises a bone marrow sample, a cord blood sample, a peripheral blood sample, or a fetal liver sample. In some embodiments, the population of cells is isolated from bone marrow of a subject subsequent to treating the subject with an amount of a mobilizing agent sufficient to mobilize the CD45^{neg} stem cells from bone marrow into the peripheral blood of the subject. As used herein, the phrase “mobilizing agent” refers to a compound (e.g., a peptide, polypeptide, small molecule, or other agent) that when administered to a subject results in the mobilization of a VSEL stem cell or a derivative thereof from the bone marrow of the subject to the peripheral blood. Stated another way, administration of a mobilizing agent to a subject results in the presence in the subjects peripheral blood of an increased number of VSEL stem cells and/or VSEL stem cell derivatives than were present therein immediately prior to the administration of the mobilizing agent. It is understood, however, that the effect of the mobilizing agent need not be instantaneous, and typically involves a lag time during which the mobilizing agent acts on a tissue or cell type in the subject in order to produce its effect. In some embodiments, the mobilizing agent comprises at least one of granulocyte-colony stimulating factor (G-CSF) and a CXCR4 antagonist (e.g., a T140 peptide; Tamamura et al. (1998) 253 Biochem Biophys Res Comm 877-882).

### IV. Methods and Compositions for Administration to Subjects

IV.A. Methods

The presently disclosed subject matter in some embodiments also provides methods for repopulating a cell type in a subject. In some embodiments, the methods comprise administering to the subject a composition comprising a plurality of isolated CD133⁺/GlyA^{neg}/CD45^{neg}/ALDH^{high} stem cells, CD133⁺/GlyA^{neg}/CD45^{neg}/ALDH^{low} stem cells, or a combination thereof in a pharmaceutically acceptable carrier in an amount and via a route sufficient to allow at least a fraction of the CD133⁺/GlyA^{neg}/CD45^{neg}/ALDH^{high} stem cells, the CD133⁺/GlyA^{neg}/CD45^{neg}/ALDH^{low} stem cells, or the combination thereof to engraft a target site and differentiate therein, whereby a cell type is repopulated in the subject. In some embodiments, the cell type is a hematopoietic cell. In some embodiments of the presently disclosed methods, the plurality of isolated CD133⁺/GlyA^{neg}/CD45^{neg}/ALDH^{high} stem cells, CD133⁺/GlyA^{neg}/CD45^{neg}/ALDH^{low} stem cells, or the combination thereof comprises CD133⁺/GlyA^{neg}/CD45^{neg}/ALDH^{high} stem cells and/or CD133⁺/GlyA^{neg}/CD45^{neg}/ALDH^{low} stem cells isolated from umbilical cord blood. In some embodiments, the target site comprises the bone marrow of the subject.

Hence, in some embodiments the presently disclosed subject matter provides methods for bone marrow transplantation. In some embodiments, the methods comprise administering to a subject with at least partially absent bone marrow a pharmaceutical preparation comprising an effective amount of CD133⁺/GlyA^{neg}/CD45^{neg}/ALDH^{high} stem cells, CD133⁺/GlyA^{neg}/CD45^{neg}/ALDH^{low} stem cells, or a combination thereof isolated from a source of said cells (e.g., cord blood, bone marrow, peripheral blood, and/or fetal liver), wherein the effective amount comprises an amount of isolated CD133⁺/GlyA^{neg}/CD45^{neg}/ALDH^{high} stem cells and/or CD133⁺/GlyA^{neg}/CD45^{neg}/ALDH^{low} stem cells sufficient to engraft in the bone marrow of the subject.

Bone marrow transplantation is a technique that generally would be well known to one of ordinary skill in the art after review of the instant disclosure. Several U.S. and other patents and patent applications have been published which describe variations on the standard technique. Briefly, a subject that will receive bone marrow transplantation (BMT) typically undergoes a series of pre-treatments that are designed to prepare the bone marrow space to receive administered cells. These pre-treatments can include, but are not limited to treatments designed to suppress the recipient's immune system so that the transplant will not be rejected if the donor and recipient are not histocompatible as well as to create space within the bone marrow to allow the administered cells to engraft. An exemplary space-creating pre-treatment comprises exposure to chemotherapeutics that destroy all or some of the bone marrow and total body irradiation (TBI).

As such, the presently disclosed subject matter provides in some embodiments a method wherein a subject with at least partially absent bone marrow has undergone a pre-treatment to at least partially reduce the bone marrow in the subject. As used herein, the phrase “a subject with at least partially absent bone marrow” refers to a subject that has received either a myeloablative treatment or a myeloreductive treatment, either of which eliminates at least a part of the bone marrow in the subject. Myeloablative and myeloreductive treatments would be known to one of ordinary skill in the art, and can include immunotherapy, chemotherapy, radiation therapy, or combinations thereof.

IV.B. Compositions

Once a subject has undergone an appropriate pre-treatment, if necessary, a composition comprising an isolated population of CD133⁺/GlyA^{neg}/CD45^{neg}/ALDH^{high} stem cells and/or CD133⁺/GlyA^{neg}/CD45^{neg}/ALDH^{low} stem cells of the presently disclosed subject matter is administered. In some embodiments, the composition comprises CD133⁺/GlyA^{neg}/CD45^{neg}/ALDH^{high} stem cells and/or CD133⁺/GlyA^{neg}/CD45^{neg}/ALDH^{low} stem cells in a pharmaceutically acceptable carrier (optionally, a carrier that is pharmaceutically acceptable for use in a human).

In some embodiments, freshly isolated CD133⁺/GlyA^{neg}/CD45^{neg}/ALDH^{high} stem cells and/or CD133⁺/GlyA^{neg}/CD45^{neg}/ALDH^{low} stem cells of the presently disclosed subject matter are administered, although frozen cells can also be employed. Methods for cryopreserving stem cells for administration to subject are known to one of ordinary skill in the art.

In some embodiments, the CD133⁺/GlyA^{neg}/CD45^{neg}/ALDH^{high} stem cells and/or CD133⁺/GlyA^{neg}/CD45^{neg}/ALDH^{low} stem cells of the presently disclosed subject matter are co-cultured in the presence of a feeder cell layer to enhance the efficiency with which the cells engraft the subject and/or produce blood cells in the subject. In some embodiments, the feeder cell layer comprises OP9 cells.

IV.B.1. Formulations

The compositions of the presently disclosed subject matter comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic methods and compositions of the presently disclosed subject matter can be used with additional adjuvants or biological response modifiers including, but not limited to, cytokines and other immunomodulating compounds.

IV.B.2. Administration

Suitable methods for administration the compositions of the presently disclosed subject matter include, but are not limited to intravenous administration and delivery directly to the target tissue or organ. In some embodiments, the method of administration encompasses features for regionalized delivery or accumulation of the cells at a target site (e.g., the bone marrow). In some embodiments, the cells are delivered directly into the target site. In some embodiments, selective delivery of the cells of the presently disclosed subject matter is accomplished by intravenous injection of cells, where they home to the target site and engraft therein.

IV.B.3. Dose

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or is treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using the assay methods described herein, one skilled in the art can readily assess the potency and efficacy of a candidate compound of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

### V. Other Applications

The presently disclosed subject matter also provides methods for inducing hematopoietic competency in CD133⁺/GlyA^{neg}/CD45^{neg}/ALDH^{high} stem cells and/or CD133⁺/GlyA^{neg}/CD45^{neg}/ALDH^{low} stem cells. As used herein, the phrase “hematopoietic competency” refers to an ability of a CD133⁺/GlyA^{neg}/CD45^{neg}/ALDH^{high} stem cell and/or CD133⁺/GlyA^{neg}/CD45^{neg}/ALDH^{low} stem cell (or a progeny cell thereof) to differentiate into a hematopoietic cell (e.g., a terminally differentiated hematopoietic cell). The phrase thus encompasses the efficiency at which an individual cell can repopulate a subject (e.g., as measured by the minimum number of cells that need to be administered to a subject in order for the subject to receive a clinically relevant benefit) as well as the time necessary for the cell to generate the clinically relevant benefit in the subject. In some embodiments, the hematopoietic competency of the cells of the presently disclosed subject matter comprises an ability to provide long term engraftment of the bone marrow in the subject.

As disclosed herein, CD133⁺/GlyA^{neg}/CD45^{neg}/ALDH^{high} stem cells and/or CD133⁺/GlyA^{neg}/CD45^{neg}/ALDH^{low} stem cells can show differing hematopoietic competencies based, in some embodiments, on the source from which the CD133⁺/GlyA^{neg}/CD45^{neg}/ALDH^{high} stem cells and/or CD133⁺/GlyA^{neg}/CD45^{neg}/ALDH^{high} stem cells were isolated, and any pre-treatment that the cells might have received (e.g., co-culture with OP9 cells). Therefore, in some embodiments the methods of the presently disclosed subject matter comprise (a) providing a CD133⁺/GlyA^{neg}/CD45^{neg}/ALDH^{high} stem cells and/or a CD133⁺/GlyA^{neg}/CD45^{neg}/ALDH^{low} stem cell; and (b) co-culturing the CD133⁺/GlyA^{neg}/CD45^{neg}/ALDH^{high} stem cell and/or CD133⁺/GlyA^{neg}/CD45^{neg}/ALDH^{low} stem cell in the presence of a feeder layer (e.g., an OP9 feeder layer) for a time sufficient to induce hematopoietic competency in the CD133⁺/GlyA^{neg}/CD45^{neg}/ALDH^{high} stem cell and/or CD133⁺/GlyA^{neg}/CD45^{neg}/ALDH^{high} stem cell.

Additionally, the presently disclosed methods can employ the CD133⁺/GlyA^{neg}/CD45^{neg}/ALDH^{high} stem cells and/or CD133⁺/GlyA^{neg}/CD45^{neg}/ALDH^{low} stem cells that are bone marrow-derived CD133⁺/GlyA^{neg}/CD45^{neg}/ALDH^{high} stem cells and/or CD133⁺/GlyA^{neg}/CD45^{neg}/ALDH^{low} stem cells; cord blood-derived CD133⁺/GlyA^{neg}/CD45^{neg}/ALDH^{high} stem cells and/or CD133⁺/GlyA^{neg}/CD45^{neg}/ALDH^{low} stem cells; or combinations thereof.

### VI. Cell Culture Systems

In some embodiments, the presently disclosed subject matter provides cell culture systems comprising CD133⁺/GlyA^{neg}/CD45^{neg}/ALDH^{high} stem cells and/or CD133⁺/GlyA^{neg}/CD45^{neg}/ALDH^{low} stem cells. In some embodiments, the cell culture systems further comprise a feeder cell layer, optionally an OP9 cell feeder layer.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Materials and Methods Employed in the Examples

Removal of Erythrocytes Using Hypotonic Lysis Before Sorting was Superior to FICOLL-PAQUE™ Centrifugation.

Removal of RBCs is a desirable step in preparing nucleated cells for staining and subsequent sorting. Therefore, two different strategies were employed to remove RBCs from UCB samples: lysis in hypotonic ammonium chloride and FICOLL-PAQUE™ centrifugation, to enrich UCB for nucleated cells and compared the percentage of CD34⁺, CXCR4⁺, and CD133⁺ cells among the Lin^{neg}/CD45^{neg} and Lin^{neg}/CD45⁺ fractions of UCB cells isolated with both methods.

Flow cytometry data revealed that the percentages of all three subpopulations of Lin^{neg}/CD45^{neg} cells (CD34⁺, CD133⁺, and CXCR4⁺) were similar among nucleated cells isolated after hypotonic lysis and after FICOLL-PAQUE™ centrifugation (0.062±0.015% vs. 0.045±0.009% for Lin^{neg}/CD45^{neg}/CD34⁺ cells, 0.013±0.001% vs. 0.022±0.004% for Lin^{neg}/CD45^{neg}/CXCR4⁺ cells, and 0.012±0.003% vs. 0.010±0.002% for Lin^{neg}/CD45^{neg}/CD133⁺ cells, respectively). On the other hand, a higher percentage of CD34⁺, CXCR4⁺, and CD133⁺ cells were observed among the nucleated Lin^{neg}/CD45⁺ fraction enriched for HSCs isolated with FICOLL-PAQUE™compared to cells obtained after hypotonic lysis (0.489±0.122% vs. 0.230±0.052% of Lin^{neg}/CD45⁺/CD34⁺ cells, 0.083±0.030% vs. 0.021±0.006% of Lin^{neg}/CD45⁺/CXCR4⁺ cells, and 0.302±0.075% vs. 0.149±0.023% of Lin^{neg}/CD45⁺/CD133⁺ cells, respectively). All flow cytometric analyses were performed following fixation and staining of cells with 7-Aminoactinomycin D (7-AAD) to exclude anucleated debris.

Next, quantitative analysis was performed and the total number of particular cell populations that could be isolated from 1 ml of UCB using both methods was calculated. First, it was determined that while 85.9±4.5% of total white blood cells present in UCB were recovered after lysis of RBCs, only 44.0±4.3% of cells were obtained after FICOLL-PAQUE™ isolation, which was expected due to the depletion of granulocytes (Zuba-Surma et al., 2010).

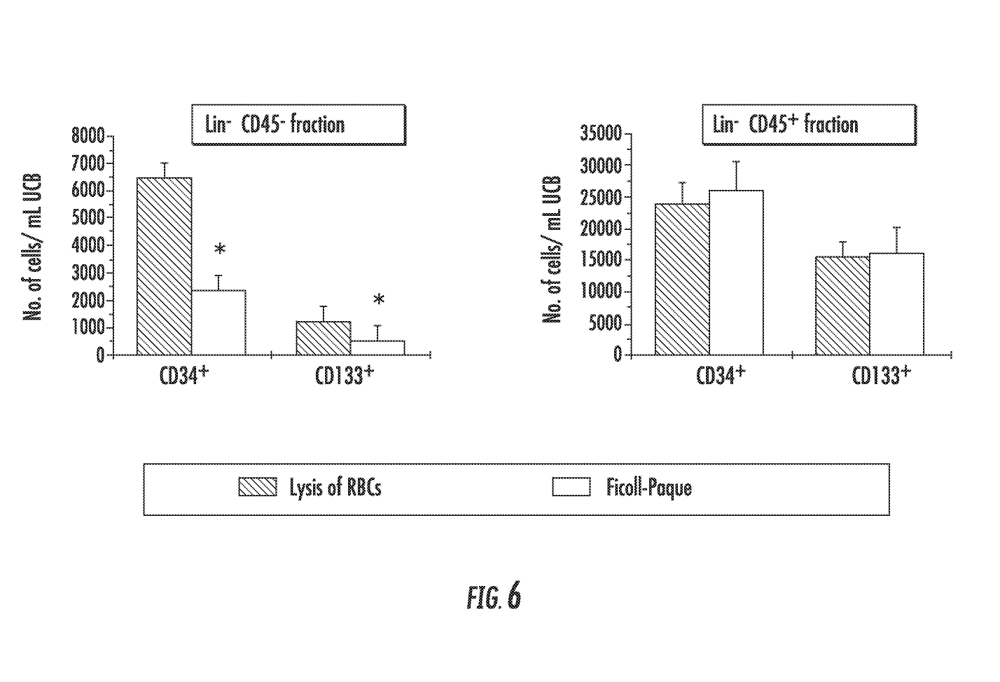

Subsequently, the percentage content of all analyzed populations was multiplied by the total number of cells obtained with each preparation method to determine the absolute number of each population per 1 ml of UCB. HSCs were effectively isolated by both methods, indicated by similar total numbers of cells obtained from 1 ml of UCB. Conversely, it was observed that the UCB subpopulations enriched for UCB-VSELs (Lin^{neg}/CD45^{neg}/CD34⁺ and Lin^{neg}/CD45^{neg}/CD133⁺) were significantly diminished during FICOLL-PAQUE™ preparation compared to hypotonic lysis (2381.6±469.6 vs. 6454.2±1564.2 cells/1 ml of UCB for Lin^{neg}/CD45^{neg}/CD34⁺ cells and 505.6±91.4 vs. 1259.7±355.1/1 ml of UCB for Lin^{neg}/CD45^{neg}/CD133⁺ cells, respectively). See FIG. 6. See also Zuba-Surma et al., 2010. This was subsequently confirmed by a decrease in expression of mRNA for genes related to pluripotency and tissue-commitment, including Oct4, Nanog, Nkx2.5/Csx, GATA-4, and VE-cadherin in MNCs, after erythrocyte depletion by FICOLL-PAQUE™ centrifugation (Zuba-Surma et al., 2010). Based on these findings, a hypotonic lysis procedure for depletion of erythrocytes was employed as described herein.

Exemplary Three-Step Procedure for Isolation and FACS Sorting of VSELs from Umbilical Cord Blood.

All experiments disclosed herein were performed in accordance with the guidelines of the local ethical and biohazard authorities at the University of Louisville School of Medicine (Louisville, Ky., United States of America). Clinical-grade UCB research units were shipped from Cleveland Cord Blood Center and were treated with 1× BD PHARM LYSE™ Buffer (BD Pharmingen, San Jose, Calif., United States of America) for 15 minutes at room temperature (RT) to remove red blood cells (RBCS) and washed twice in phosphate-buffered saline (PBS). A single-cell suspension of total nucleated cells (TNCs) obtained from clinical UCB samples was treated with antibodies against CD133 antigen-coated immunomagnetic beads and separate by using a MACS Separator (Miltenyi Biotec GMBH, Germany) to reduce cell numbers prior to cell sorting. The CD133-positive cell fraction was reacted with the ALDEFLUOR®™ ALDH detection reagent (STEMCELL™ Technologies, Vancouver, British Columbia, Canada) for detecting ALDH activity levels. After the ALDH enzyme reaction, cells were washed and resuspended in cold ALDEFLUOR® buffer (STEMCELL™ Technologies) and maintained on ice during all subsequent manipulations. Cells were incubated with phycoerythrin (PE)-conjugated murine anti-human CD235a/GlyA (clone GA-R2, BD Biosciences, San Jose, Calif., United States of America), phycoerythrin-CY7 (PE-CY7)-CD45 (clone HI30, BD Biosciences), and allophycocyanin (APC)-conjugated CD133/2 (Miltenyi Biotec GMBH, Germany). Cells were washed and resuspended in cold ALDEFLUOR® buffer and sorted by MOFLO™ sorter (Dako, Carpinteria, Calif., United States of America) to obtain populations enriched in VSELs (CD45^{neg}/GlyA^{neg}/CD133⁺/ALDH^{high} and CD45/GlyA^{neg}/CD133⁺/ALDH^{low}), as well as for hematopoietic stem/progenitor cells (HSPCs, CD45/GlyA^{neg}/CD133⁺/ALDH^{high} and CD45⁺/GlyA^{neg}/CD133⁺/ALDH^{low} cells).

Ex Vivo Differentiation of VSELs into Hematopoietic Cells in Primary Co-Cultures Over OP9 Stromal Cells.

Freshly sorted CD45^{neg}/GlyA^{neg}/CD133⁺/ALDH^{high} and CD45^{neg}/GlyA^{neg}/CD133⁺/ALDH^{low} subpopulations of VSELs and CD45⁺/GlyA^{neg}/CD133⁺/ALDH^{high} and CD45⁺/GlyA^{neg}/CD133⁺/ALDH^{low} subpopulations of hematopoietic stem/progenitor cells (HSPCs) were plated over OP9 cells in α-MEM with 20% FBS (MOLECULAR PROBES®, INVITROGEN™, a division of Life Technologies Corporation, Carlsbad, Calif., United States of America) for 7 days and subsequently trypsinized, washed by centrifugation in α-MEM, and replated in methylcellulose-based medium (STEMCELL™ Technologies).

Evaluation of the Clonogenic Potential of Sorted Cells in Methylcellulose Cultures.

VSELs or HSPCs freshly isolated from BM or cells harvested from OP9 cultures were plated in methylcellulose-based medium (STEMCELL™ Technologies) supplemented with murine stem cell growth factor (SCF), interleukin-3 (IL-3), granulocyte-macrophage colony-stimulating factor (GM-CSF), FLT3, thrombopoietin (TpO), erythropoietin (EpO), and insulin-like growth factor-2 (IGF-2). Cells were cultured for 10 days and the numbers of colonies formed were scored. Subsequently, methylcellulose cultures were solubilized and trypsinized and the resulting cells were washed by centrifugation in α-MEM and plated into secondary methylcellulose cultures. Cells were grown in the presence of the same growth factors and replated after 10 days into new methylcellulose cultures.

PCR Analysis of Gene Expression in Freshly Sorted Cells and OP9-Expanded Cells.

Total RNA (from samples of approximately 20,000 cells each) was isolated using the RNEASY® Mini Kit (Qiagen Inc., Valencia, Calif., United States of America) and genomic DNA removed using the DNA-Kit (Applied Biosystems, Foster City, Calif., United States of America). Isolated mRNA was reverse-transcribed with TAQMAN® Reverse Transcription Reagents (Applied Biosystems), according to the manufacturer's instructions. RT-PCR was performed using AMPLITAQ GOLD® (Applied Biosystems) with 1 cycle of 8 minutes at 95° C.; 2 cycles of 2 minutes at 95° C., 1 minute at 62° C., and 1 minute at 72° C.; 38 cycles of 30 seconds at 95° C., 1 minute at 62° C., and 1 minute at 72° C.; and 1 cycle of 10 minutes at 72° C. using sequence-specific primers. Quantitative measurement of target transcript expression was performed by RQ-PCR using an ABI PRISM® 7500 Sequence Detection System (Applied Biosystems). Complementary cDNA (cDNA) from indicated cells was amplified using SYBR® Green PCR Master Mix (Applied Biosystems) and specific primers. All primers were designed with PRIMER EXPRESS® software (Applied Biosystems), with at least one primer in each pair containing an exon-intron boundary. The threshold cycle (Ct) was determined and relative quantification of the expression level of target genes was obtained with the 2^{negΔΔCt} method, using β2-microglobulin (β2 mg) as an endogenous control gene and mononuclear cell (MNC) genes as calibration controls. All primers used in RT-PCR and real-time quantitative PCR (RQ-PCR) are listed in Table I and Table II, respectively.

TABLE I
Sequences of Primers Employed for
Qualitative RT-PCR Analyses

| Gene | Primer Sequences |
|---|---|
| SCL/TAL1; | F: GGCTTTGTGTGAAGGCAGAGA |
| NM_003189 | (SEQ ID NO: 1) |
| | R: TCGCCAGCATGAACAGTGAT |
| | (SEQ ID NO: 2) |
| C-myb; | F: TGCCAATTATCTCCCGAATCG |
| NM_005375 | (SEQ ID NO: 3) |
| | R: AACAGACCAACGTTTCGGACC |
| | (SEQ ID NO: 4) |
| HOXB-4; | F: ACGGTAAACCCCAATTACGC |
| NM_024015 | (SEQ ID NO: 5) |
| | R: TGTCAGGTAGCGGTTGTAGTGA |
| | (SEQ ID NO: 6) |
| LMO-2; | F: GTGTGCGAACAGGACATCTACG |
| NM_005574 | (SEQ ID NO: 7) |
| | R: AGACGGCGTCTTCAGTGAACA |
| | (SEQ ID NO: 8) |
| Oct-4/POU5F1; | F: GAGCCCTGCACCGTCACC |
| NM_002701 | (SEQ ID NO: 9) |
| | R: TTGATGTCCTGGGACTCCTCC |
| | (SEQ ID NO: 10) |
| β-Actin; | F: CGATCCACACGGAGTACTTG |
| NM_001101 | (SEQ ID NO: 11) |
| | R: GGATGCAGAAGGAGATCACTG |
| | (SEQ ID NO: 12) |

In Tables I and II, locus names are presented in bold in the first column and exemplary GENBANK® database Accession Nos. that correspond to nucleic acid gene products derived from the listed human loci are presented in regular font text immediately below the locus names. In the second column, F indicates the forward primer sequences and R indicates the reverse primer sequences for each locus. Sequences are provided in 5′ to 3′ orientation for each primer.

TABLE II
Sequences of Primers Employed for
RT-PCR Analysis

| Gene | Primer Sequences |
|---|---|
| SCL/TAL1 | F: GTTCACCACCAACAATCGAGTG |
| NM_003189 | (SEQ ID NO: 13) |
| | R: GATATACTTCATGGCCAGGCG |
| | (SEQ ID NO: 14) |
| C-myb; | F: GCCAATTATCTCCCGAATCGA |
| NM_005375 | (SEQ ID NO: 15) |
| | R: TTCGTCCAGGCAGTAGCTTTG |
| | (SEQ ID NO: 16) |
| HOXB-4, | F: ACGGTAAACCCCAATTACGCC |
| NM_024015 | (SEQ ID NO: 17) |
| | R: TTTTCCACTTCATGCGCCG |
| | (SEQ ID NO: 18) |
| LMO-2; | F: CCCTTCAGAGGAACCAGTGGAT |
| NM_005574 | (SEQ ID NO: 19) |
| | R: CTTTCACCCGCATTGTCATCTC |
| | (SEQ ID NO: 20) |
| Oct-4/POU5F1; | F: GATGGCGTACTGTGGGCCC |
| NM_002701 | (SEQ ID NO: 21) |
| | R: TTGATGTCCTGGGACTCCTCC |
| | (SEQ ID NO: 22) |
| β-Actin; | F: CGATCCACACGGAGTACTTG |
| NM_001101 | (SEQ ID NO: 23) |
| | R: GGATGCAGAAGGAGATCACTG |
| | (SEQ ID NO: 24) |

FACS Analysis of OP9-Expanded Cells.

Cells cultured over OP9 were plated in methylcellulose to grow hematopoietic colonies. Subsequently, colonies were solubilized and evaluated by FACS (LSRII, BD Biosciences) for expression of CD45, CD14, GlyA, CD3, CD19, and CD41. For detection of antigens, phycoerythrin-CY7 (PE-CY7)-conjugated murine anti-human antibody were employed for CD45 (clone HI30) and fluorescein-conjugated anti-human lineage marker antibodies were employed for CD14 (clone M5E2), GlyA (clone GLA-R2), CD3 (clone UCHT1), CD19 (clone HIB19), and CD41 (clone HIP2). AH antibodies were obtained from BD Biosciences (San Jose, Calif., United States of America).

Hematopoietic Transplantation Studies.

For transplantation experiments, NOD/SCID mice were irradiated with a sub-lethal dose of γ-irradiation (350 cGy). After 24 hours, freshly isolated VSELs, HSCs, or OP9-primed/expanded cells were transplanted into mice by tail vein. Anesthetized transplanted mice were sacrificed 6 weeks after transplantation to evaluate chimerism in BM, PB, and spleen. For this analysis the same anti-human antibodies that are listed above were employed.

Statistical Analysis.

All data in quantitative (q)ChIP and gene expression analyses were analyzed using one-factor Analysis of Variance (ANOVA) with Bonferroni's Multiple Comparison Test. The Instat1.14 program (GraphPad Software, Inc., La Jolla, Calif., United States of America) was employed, and statistical significance was defined as p<0.05 or p<0.01.

### Example 1

### ALDH Activity as a New Approach to Purifying CD133⁺/GlyA^{neg}/CD45^{neg} UCB VSELs

UCB-VSELs were initially purified from erythrocyte-depleted UCB by multiparameter sorting for a population of CD133⁺/CD45^{neg}/Lin^{neg} cells. This procedure, however, is relatively time consuming and the multiparameter sorting time required to process one entire cord blood unit (about 50-100 ml) to isolate rare VSELs from UCB MNCs typically takes 3-4 days.

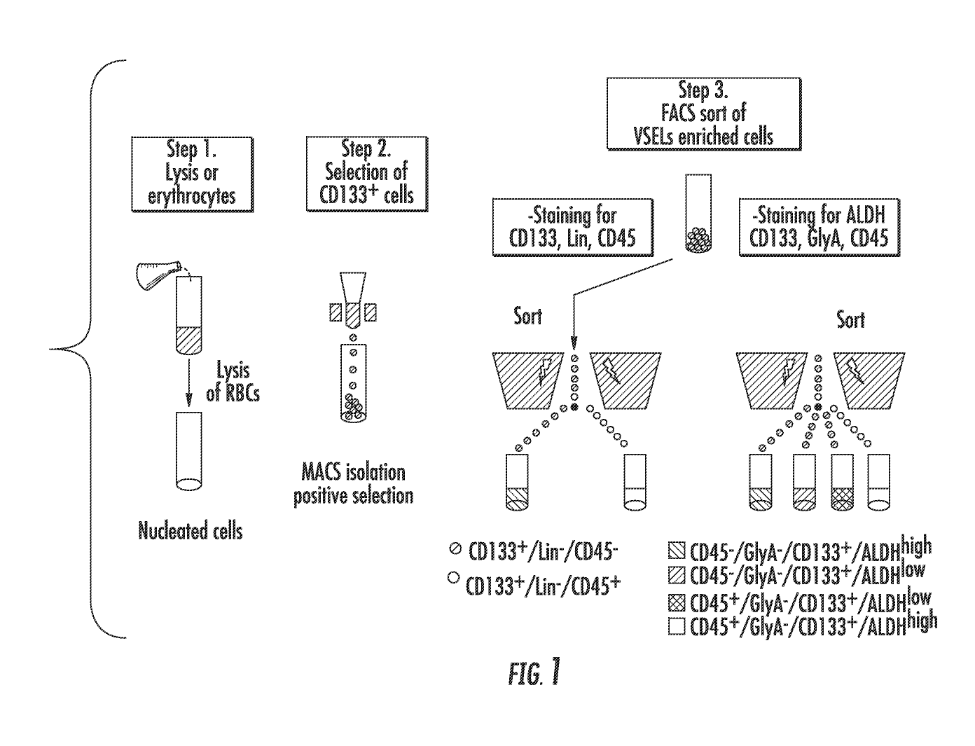

In order to establish a more efficient method for VSEL purification from UCB, a three-step isolation strategy was employed (see FIG. 1) based on removal of erythrocytes/RBCs by hypotonic lysis (in some embodiments, a 1^{st} step), immunomagnetic separation of CD133⁺ cells (in some embodiments, a 2^{nd} step), followed by FACS-based isolation of CD133⁺/GlyA^{neg}/CD45^{neg} cells (in some embodiments, a 3^{rd} step). The rationale for using lysis buffer to remove erythrocytes was based on an observation that doing so leads to a higher yield of VSELs than removal of erythrocytes by centrifugation over a FICOLL-PAQUE™ gradient (Zuba-Surma et al., 2010). On the other hand, the selection of CD133 antigen for VSEL isolation was based on an observation by the co-inventors that CD133⁺ VSELs were highly enriched for pluripotent stem cell transcription factor expression (e.g., Oct-4 and SSEA-4). See Zuba-Surma et al., 2010. Inclusion of an anti-GlyA antibody was based on the fact that small erythroblasts that are GlyA⁺ and are present in UCB do not express CD45 antigen. Thus, selection for CD45^{neg} cells was used to enrich for these cells.

Additionally, the cells were also FACS-sorted after exposure to ALDEFLUOR®™ ALDH detection reagent, which permitted the further separation of CD133⁺/GlyA^{neg}/CD45^{neg} cells into ALDH^{high} and ALDH^{low} subpopulations.

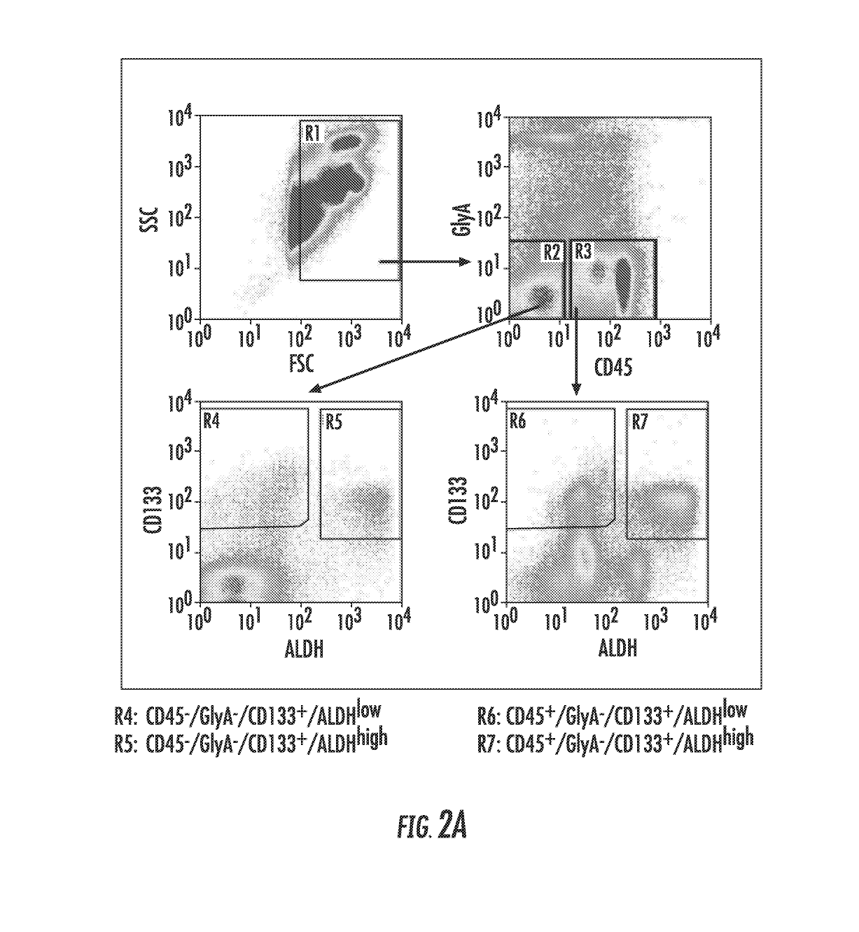

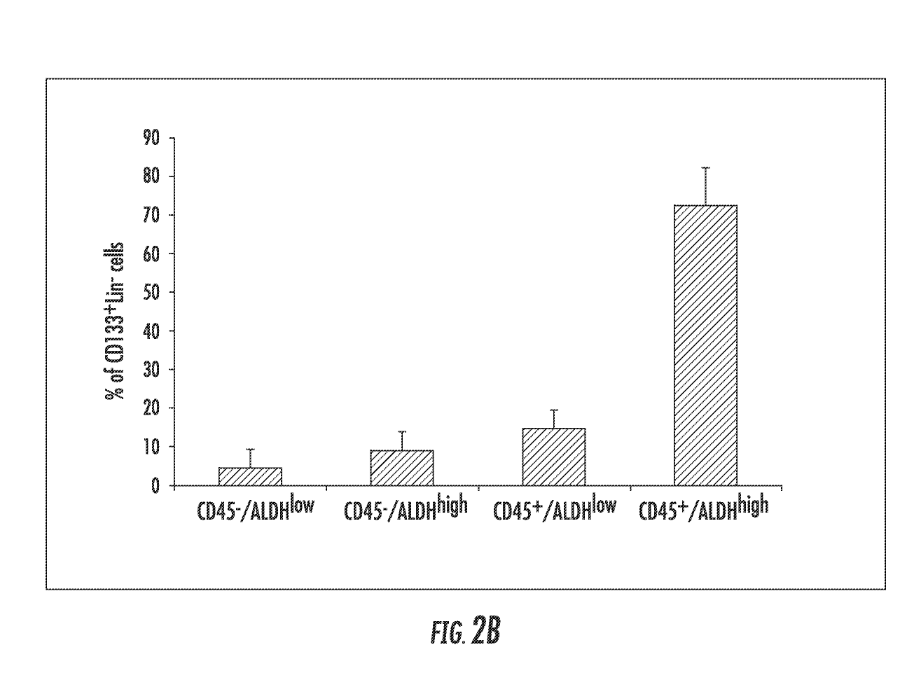

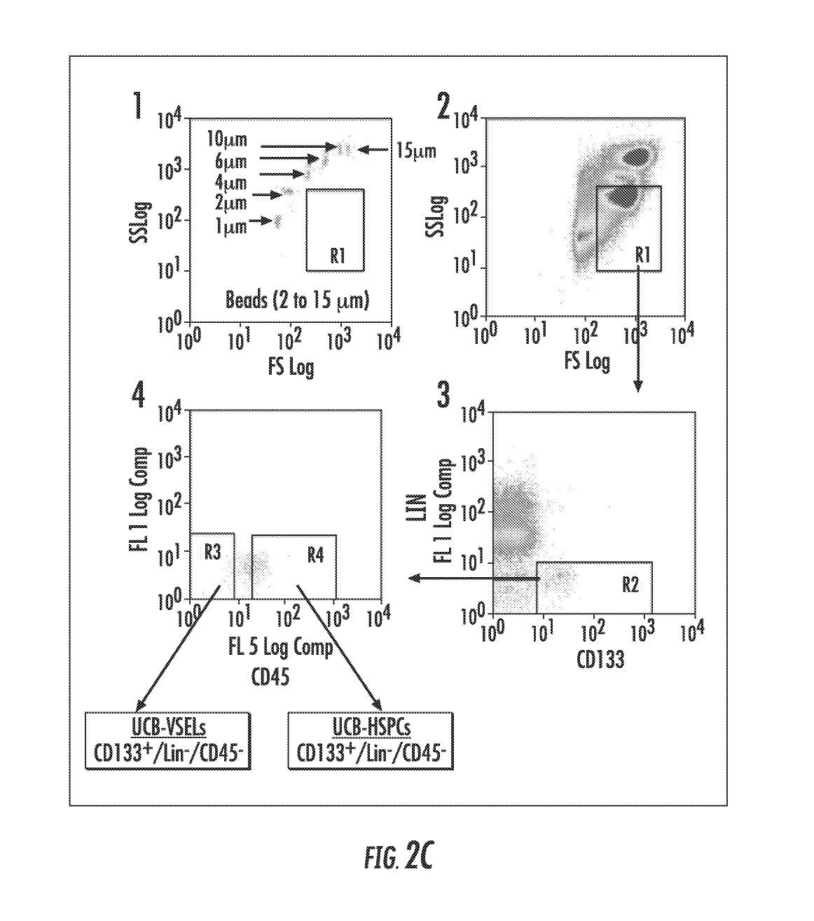

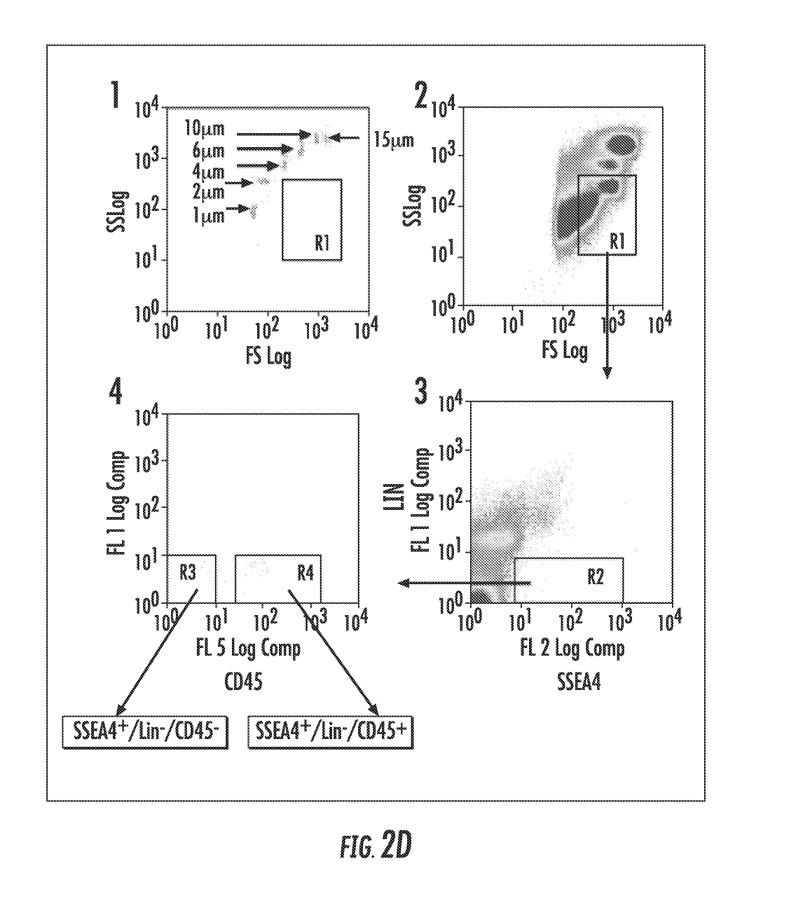

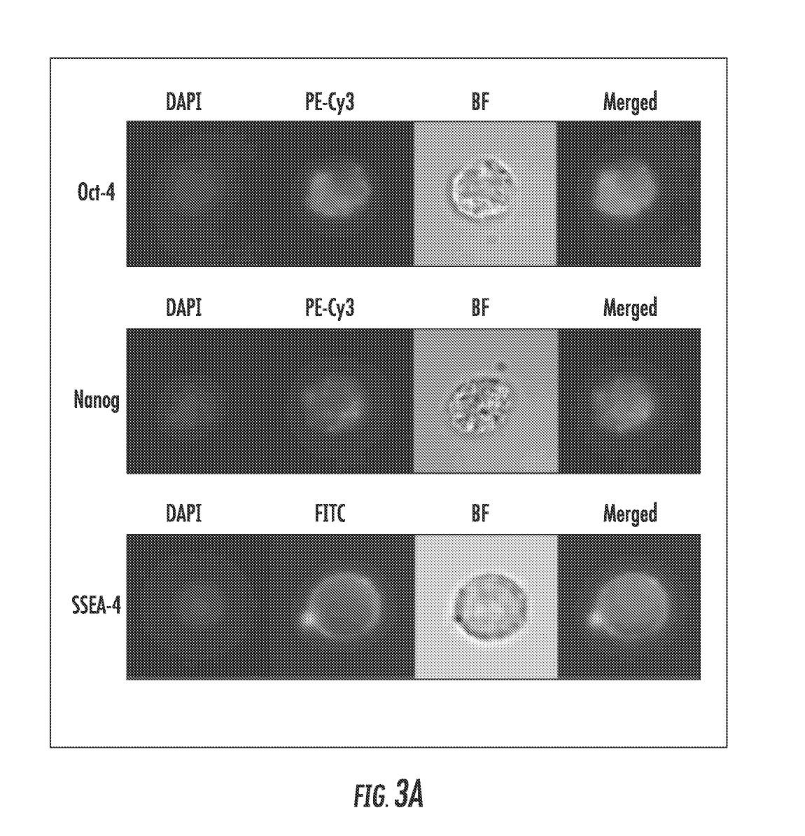

FIG. 2A shows the isolation strategy of immunomagnetic-separated UCB CD133⁺ cells based on ALDH activity and CD133, GlyA, and CD45 expression. By employing this strategy, it was possible to enriched for VSELs (CD45^{neg}/GlyA^{neg}/CD133⁺/ALDH^{high} and CD45/GlyA^{neg}/CD133⁺/ALDH^{low}) as well as for HSPCs (CD45⁺/GlyA^{neg}/CD133⁺/ALDH^{high} and CD45⁺/GlyA^{neg}/CD133⁺/ALDH^{low}). FIG. 2B shows these cell fractions as a percentage of total CD133⁺/GlyA^{neg} UCB cells. The fractions of CD133⁺ cells enriched for CD45^{neg}/GlyA^{neg}/CD133⁺/ALDH^{low} and CD45^{neg}/GlyA^{neg}/CD133⁺/ALDH^{high} VSELs are the smallest ones (˜5% and ˜10%, respectively). The majority of CD133⁺/GlyA^{neg} cells were CD45⁺/GlyA^{neg}/CD133⁺/ALDH^{high} HSPCs (˜70%) followed by CD45⁺/GlyA^{neg}/CD133⁺/ALDH^{low} HSPCs (˜15%). The total number of these rare cells per 100 ml of UCB is shown in Table III. Both CD45⁺/GlyA^{neg}/CD133⁺/ALDH^{high} and CD45⁺/GlyA^{neg}/CD133⁺/ALDH^{low} VSELs are 2-3 orders of magnitude less numerous than the CD45⁺ fractions of HSPCs.

Alternatively, FIG. 2C shows exemplary gating strategies for FACS sorting of UCB VSELs and HSCs based on expression of CD133, CD45, and lineage markers. UCB-VSELs were isolated from fraction of human UCB total nucleated cells (TNCs) by FACS by employing following gating criteria. In FIG. 20, panel 1, all events ranging from 2 μm are included in gate R1 after comparison with six differently sized bead particles with standard diameters of 1, 2, 4, 6, 10 and 15 μm. In FIG. 2C, panel 2, UCB-derived TNCs are visualized on a dot plot based on FSC vs. SSC signals. In FIG. 20, panel 3, cells from region R1 are further analyzed for CD133 and Lin expression: Lin^{neg}/CD133⁺ events are included in region R2. In FIG. 2C, panel 4, the Lin^{neg}/CD133⁺ population from region R2 is subsequently analyzed based on CD45 antigen expression and CD45^{neg} and CD45⁺ subpopulations visualized on dot plot, i.e., CD133⁺/Lin^{neg}/CD45^{neg} (VSELs: region R3) and CD133⁺/Lin^{neg}/CD45⁺ (HSCs: region R4).

TABLE III
Total Number of VSELs per 100 ml of UCB

| | Total number of cells per |
|---|---|
| Phenotype of cells | 100 ml of UCB |
| CD45^{neg}/GlyA^{neg}/CD133⁺/ALDH^{high} | ~10³ (± 0.3) |
| CD45^{neg}/GlyA^{neg}/CD133⁺/ALDH^{low} | ~3.6 × 10³ (± 0.8) |
| CD45⁺/GlyA^{neg}/CD133⁺/ALDH^{low} | ~46 × 10³ (± 2.0) |
| CD45⁺/GlyA^{neg}/CD133⁺/ALDH^{high} | ~375 × 10³ (± 12.0) |

### Example 2

### SSEA-4 as a Positive Marker for UCB-VSEL Purification by FACS

Immunohistochemical staining experiments indicated that UCB-VSELs highly expressed SSEA-4 on their cell surface. An antibody against SSEA-4 that was recommended by Dr. Yoshiaki Sonoda (Kansai University, Osaka, Japan) was employed for FACS sorting, which facilitated the purification of UCB-VSELs as a population of SSEA-4⁺/Lin^{neg}/CD45^{neg} cells (see FIG. 2D).

FIG. 2D is a series of FACS scatter plots depicted exemplary gating strategies for FACS sorting of UCB VSELs and HSCs based on expression of SSEA-4, CD45, and lineage markers. SSEA-4⁺/Lin^{neg}/CD45^{neg} cells were isolated from fraction of human UCB TNCs by FACS by employing following gating criteria. In FIG. 2D, panel 1, all events ranging from 2 μm are included in gate R1 after comparison with six differently sized bead particles with standard diameters of 1, 2, 4, 6, 10 and 15 μm. In FIG. 20, panel 2, UCB-derived TNCs are visualized on a dot plot based on FSC vs, SSC signals. In FIG. 2D, panel 3, cells from region R1 are further analyzed for SSEA-4 and Lin expression: Lin^{neg}/SSEA-4⁺ events are included in region R2. In FIG. 2D, panel 4, the Lin^{neg}/SSEA-4⁺ population from region R2 was subsequently analyzed based on CD45 antigen expression and CD45^{neg} and CD45⁺ subpopulations visualized on dot plot (i.e., SSEA-4⁺/Lin^{neg}/CD45^{neg}(region R3) and SSEA-4⁺/Lin^{neg}/CD45⁺; region R4).

### Example 3

### Molecular Characterization of Sorted Cells

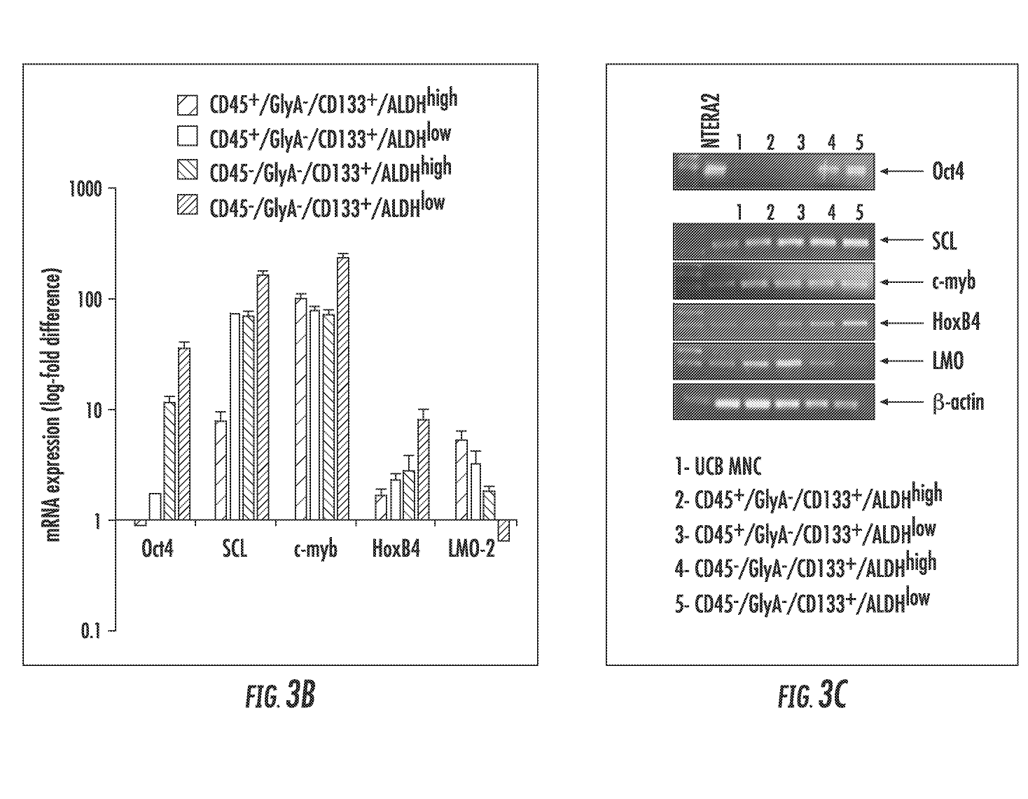

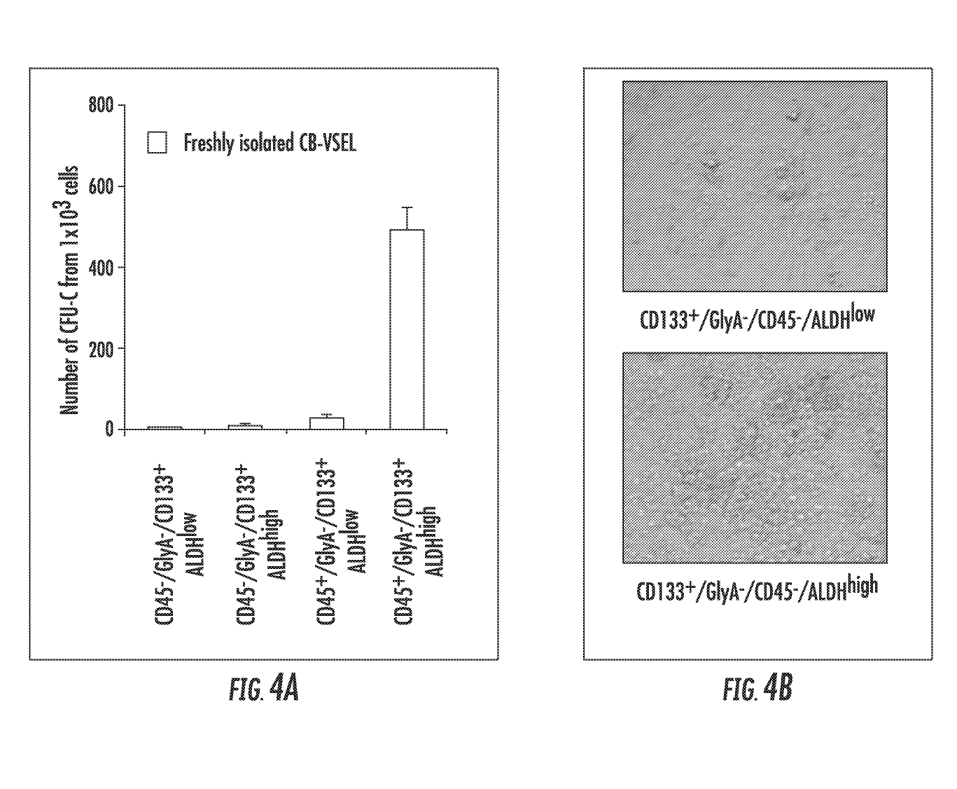

The presence of Oct-4, Nanog, and SSEA-1 proteins in UCB-sorted CD45^{neg}/Gly-A^{neg}/CD133⁺/ALDH^{low} VSELs was confirmed by immunohistochemical staining (see FIG. 3A). Next, RT-PCR analysis of gene expression confirmed that CD45^{neg}/GlyA^{neg}/CD133⁺/ALDH^{low} VSELs have the highest expression of Oct-4, Scl-2, and HoxB4 (see FIGS. 3B and 3C). In contrast, the expression of the LMO2 gene (LIM domain only 2 (rhombotin-like 1)) was highest in CD45⁺/GlyA^{neg}/CD133⁺/ALDH^{high} HSPCs. Interestingly, freshly sorted human VSELs and HSCs expressed similar levels of c-myb.

### Example 4

### Clonogenic Potential In Vitro of UCB-Derived VSELs

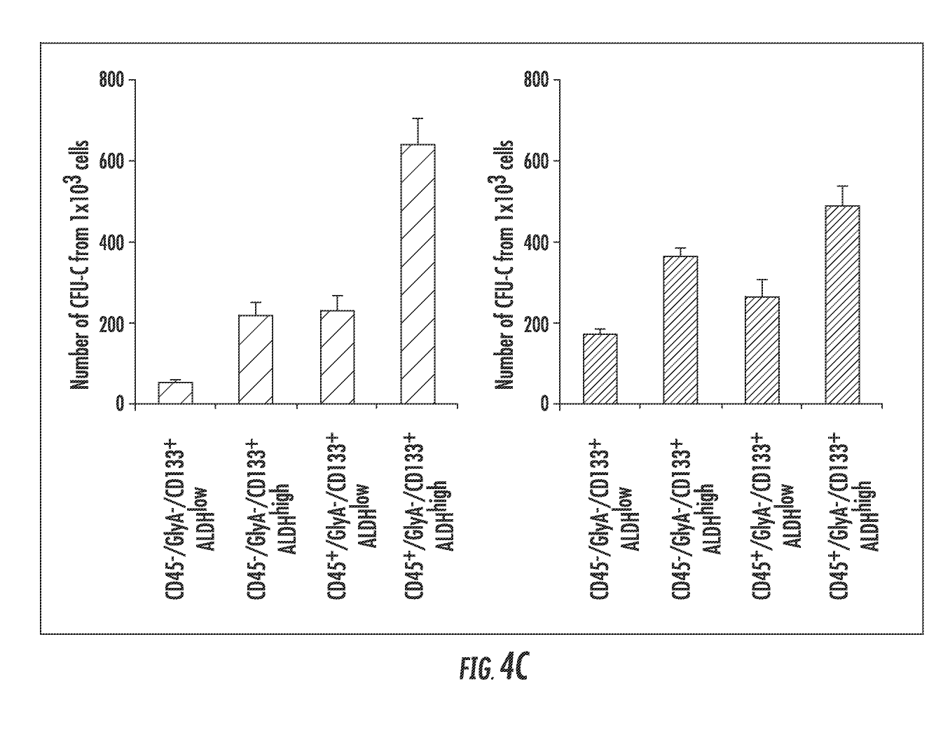

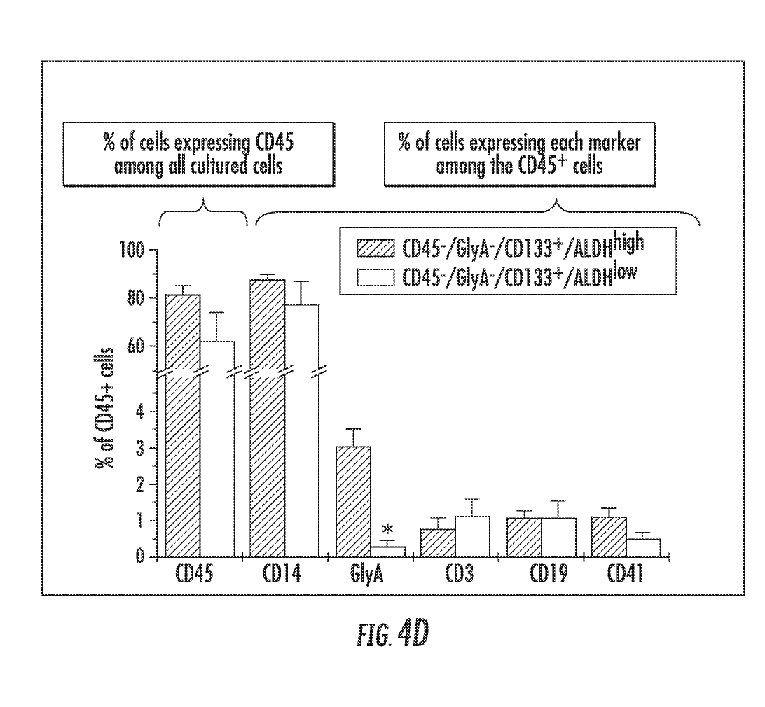

After sorting by FACS from UCB, CD45^{neg}/GlyA^{neg}/CD133⁺/ALDH^{high}, CD45^{neg}/GlyA^{neg}/CD133⁺/ALDH^{low}, CD45⁺/GlyA^{neg}/CD133⁺/ALDH^{high}, and CD45⁺/GlyA^{neg}/CD133⁺/ALDH^{low} cells were subsequently plated in METHOCULT® methylcellulose-based culture medium supplemented with a cocktail of cytokines and growth factors promoting growth of clonogenic colonies (CFU-C). It was observed that while CD45⁺/GlyA^{neg}/CD133⁺/ALDH^{high} cells freshly isolated from UCB had the highest clonogenic potential, CD45^{neg}/GlyA^{neg}/CD133⁺/ALDH^{low} cells did not grow colonies at all. The clonogenic potential of CD45^{neg}/GlyA^{neg}/CD133⁺/ALDH^{high} and CD45⁺/GlyA^{neg}/CD133⁺/ALDH^{high} cells was very low (FIG. 4A).

Like ES cells and iPS cells, murine VSELs must be co-cultured/primed over OP9 stroma in order to acquire hematopoietic commitment (Ratajczak et al., 2011). Therefore, a similar strategy was employed for human VSELs. It was found that after 7 days, UCB-purified VSELs (CD45^{neg}/GlyA^{neg}/CD133⁺/ALDH^{low} and CD45^{neg}/Gly-A^{neg}/CD133⁺/ALDH^{high}) plated over OP9 cells formed colonies resembling cobblestones (see FIG. 4B). Of note, the ability to form cobblestone areas was lower for ALDH^{low} VSELs.

In the next step, cells from these cultures were trypsinized on day 7 and subsequently tested for clonogenic potential in METHOCULT® methylcellulose-based culture media (STEMCELL™ Technologies). FIG. 4C (left panel) shows the number of colonies formed by cells isolated from OP9 stroma cultures that were initiated by all four fractions of sorted UCB cells. The number of plated cells was adjusted to have the same numbers of human cells. A significant increase in the clonogenic potential of UCB-VSELs was observed, in particular, in a population of cells that was initiated by CD45^{neg}/GlyA^{neg}/CD133⁺/ALDH^{high} VSELs; however, colonies were also formed by cells initiated from CD45^{neg}/GlyA^{neg}/CD133⁺/ALDH^{low} VSELs. The clonogenic potential of cells derived from cultures initiated by UCB VSELs additionally increased 10 days after they were plated into secondary methylcellulose cultures (see FIG. 4C; right panel), which suggested that these cells need more time to differentiate along the hematopoietic lineage. Of note, colonies grown in these cultures from VSEL-derived CD45⁺ cells were 2-3 times larger than those initiated by HSPC-expanded cells.

FACS analysis of cells derived from aspirated methylcellulose hematopoietic colonies initiated by VSEL-derived cells revealed that cells in these colonies highly expressed CD45, CD14, CD3, CD19, and CD41 antigens (see FIG. 40). Interestingly, cells expanded from CD45^{neg}/CD133⁺/ALDH^{high} VSELs were significantly enriched for GlyA⁺ erythroid precursors.

### Example 5

### UCB-VSELs Expanded Over OP9 Cells are Able to Engraft NOD/SCID Immunodeficient Animals

The in vivo hematopoietic potential of UCB-derived VSELs expanded over OP9 cultures was tested after transplantation into immunodeficient, sublethally irradiated NOD/SCID mice. However, because no chimerism was observed when freshly sorted, non-OP9-cultured freshly purified VSELs were transplanted, OP9 primed/expanded cells were transplanted.

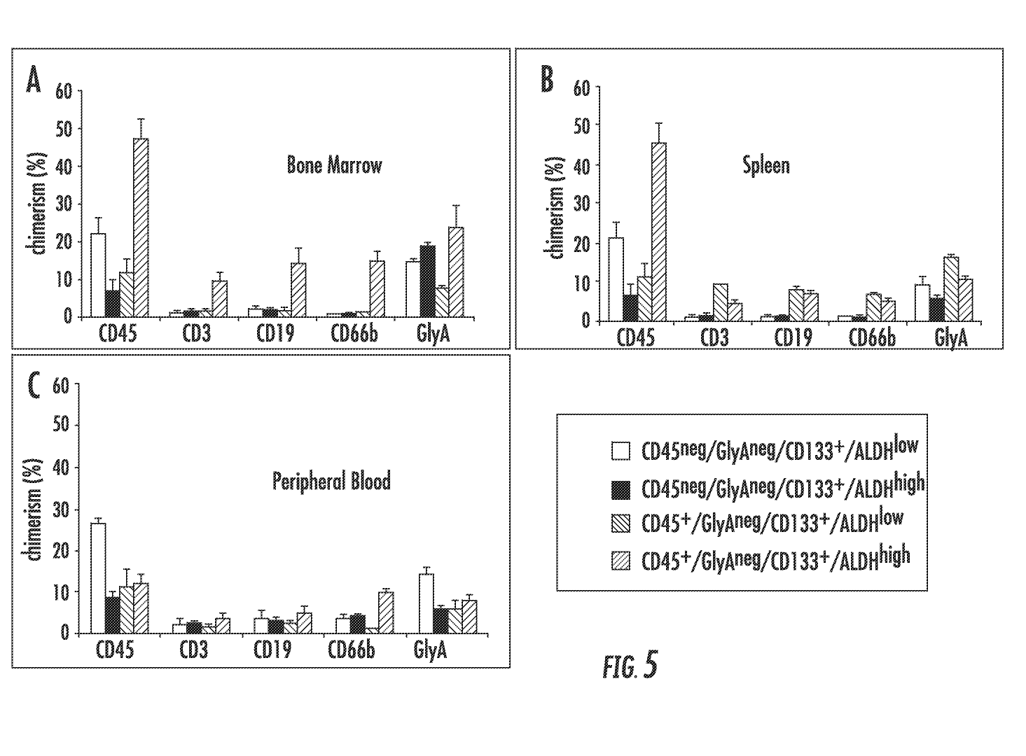

OP9-primed cultures initiated by the same number of sorted cells were trypsinized after 7 days and cells were injected intravenously. After 6 weeks, mice were sacrificed and human-murine chimerism was evaluated in all major hematopoietic lineages in BM, spleen, and peripheral blood by FACS using human-specific antibodies. The highest level of chimerism in BM and spleen in all hematopoietic lineages was achieved after transplantation of CD45⁺/CD133⁺/ALDH^{high} HSPCs (see FIG. 5). However, significant chimerism was also observed after transplantation of OP9-cultured VSELs.

### Example 6

### UCB-VSELs are Lost During Routine Volume Depletion in UCB Banking

Because of the small size of UCB-VSELs and their different density due to a high nuclear/cytoplasmic ratio, there is the possibility that they could be depleted at various steps proceeding sorting. Therefore, whether volume depletion before freezing in blood banking is a step where UCB-VSELs could be lost was tested. To address this issue, the content of Lin^{neg}/CD45⁺/CD34⁺ and Lin^{neg}/CD45⁺/CD133⁺ UCB-VSELs and their CD45⁺ hematopoietic counterparts was tested under the following conditions: (i) in fresh UCB samples before processing; (ii) in concentrates of these cells prepared for freezing by volume depletion with the AXP™ AutoXpress Platform; and (iii) in UCB samples after thawing.

Flow cytometric analysis revealed a significant loss of total nucleated CD34⁺ and CD133⁺ cells, as well as Lin^{neg}/CD45^{neg}/CD34⁺, Lin^{neg}/CD45⁺/CD34⁺, Lin^{neg}/CD45^{neg}/CD133⁺, and Lin^{neg}/CD45⁺/CD133⁺ cells in the concentrates of UCB cells processed and prepared for frozen storage when employing the volume-depletion strategy (tuba-Surma et al., 2010). It was determined that an average of 41.5±15.9% of Lin^{neg}/CD45^{neg}/CD34⁺ and 42.5±12.6% of Lin^{neg}/CD45^{neg}/CD133⁺ cells were lost during such procedures. At the same time, the loss of only 26.9±14.8% of Lin^{neg}/CD45⁺/CD34⁺ and 26.7±12.5% of Lin^{neg}/CD45⁺/CD133⁺ cells that are enriched for HSPCs was observed (Zuba-Surma et al., 2010). Interestingly, it was also observed that UCB-derived VSELs are somewhat more resistant to the freeze-thaw procedure than HSPCs.

### Discussion of the Examples

As disclosed herein, a population of CD133⁺/lin^{neg}/CD45^{neg} very small embryonic-like stem cells (VSELs) was purified by multiparameter sorting from umbilical cord blood (UCB). In order to speed up isolation of these cells, anti-CD133-conjugated paramagnetic beads followed by staining with ALDEFLUOR® ALDH detection reagent to detect ALDH activity was employed. Subsequently, CD45^{neg}/GlyA^{neg}/CD133⁺/ALDH^{high} and CD45^{neg}/GlyA^{neg}/CD133⁺/ALDH^{low} cells (which were enriched for VSELs) and CD45⁺/GlyA/CD133⁺/ALDH^{high} and CD45⁺/GlyA^{neg}/CD133⁺/ALDH^{low} cells (which were enriched for HSPCs) were sorted. While freshly isolated CD45^{neg} VSELs did not grow hematopoietic colonies when plated alone, the same cells acquired hematopoietic potential when activated/expanded over OP9 stromal feeder cells, and grew colonies containing CD45⁺ hematopoietic cells in methylcellulose cultures.

It was also observed that CD45^{neg}/GlyA^{neg}/CD133⁺/ALDH^{high} VSELs grew colonies earner than CD45^{neg}/GlyA^{neg}/CD133⁺/ALDH^{low} VSELs, which suggested that the latter cells might need more time to acquire hematopoietic commitment. In support of this possibility, real-time PCR analysis confirmed that, while freshly isolated CD45^{neg}/GlyA^{neg}/CD133⁺/ALDH^{high} VSELs expressed more hematopoietic transcripts (e.g., c-myb), CD45^{neg}/GlyA^{neg}/CD133⁺/ALDH^{low} VSELs exhibited higher levels of pluripotent stem cell markers (e.g., Oct-4). Additionally, hematopoietic cells derived from VSELs that were co-cultured over OP9 support were able to establish human lympho-hematopoietic chimerism in lethally irradiated NOD/SCID mice 4-6 weeks after transplantation. These observations suggested that VSELs (e.g., UCB-VSELs) corresponded to the most primitive population of HSPCs.

The phenotype of the most primitive human LT-HSCs is still not very well defined and several potential candidate cells have been proposed based on the expression of cell-surface antigens (e.g., CD133⁺, CD34⁺, CD38^{neg}, and Lin^{neg}), SLAM markers, and low levels of staining by some fluorescent dyes (e.g., Rh123^{dull}, Pyronin and Hoechst 33342^{low}). See Kiel et al., 2005; Ratajczak, 2008.

A useful detection system for identification of primitive HSPCs is exposure of cells to ALDEFLUOR® ALDH detection reagent (STEMCELL™ Technologies) to detect the presence of ALDH biological activity (Hess et al., 2008). The ALDEFLUOR® detection reagent is a substrate for ALDH, a cytosolic enzyme highly expressed in less-differentiated hematopoietic cells and implicated in resistance to some alkylating agents (Hess et al., 2008). In the presence of ALDH, the ALDEFLUOR® detection reagent becomes modified to the fluorescent molecule that marks ALDH-expressing cells. The ALDEFLUOR® detection reagent-based staining can be combined with other stem cell markers, for example, CD133 antigen. It was reported that human ALDH^{high}/Lin^{neg} cells, when transplanted into immunodeficient NOD/SCID mice, robustly reconstituted hematopoiesis (Hess et al., 2004). Similar cells were recently also found among populations of human BM-derived CD133⁺/lin^{neg}/ALDH^{high} cells (Hess et al., 2006).

On the other hand, evidence is accumulating that human hematopoietic tissues (BM or UCB) might contain some rare, primitive hematopoietic stem cells that do not match the phenotype of classical HSCs and do not exhibit in vitro hematopoietic activity immediately after purification (Bhatia et al., 1998). Accordingly, it has been demonstrated that human BM contains a population of rare CD34^{neg}/Lin^{neg}/CD38^{neg} HSCs that show poor clonogenic activity in vitro, but in vivo engraft robustly in immunodeficient mice (Bhatia et al., 1998). Similarly, it has been reported that human UCB contains rare, primitive CD34^{neg}/flt^{neg}/Lin^{neg} cells that, in contrast to normal adult HSCs, do not engraft after intravenous injection and exhibit hematopoietic potential only after intra-bone delivery (Kimura et al., 2007). More recently, these cells were found to be highly enriched in a CD34^{neg} fraction of UCB cells that were depleted of differentiated cells by a cocktail of antibodies against eighteen different lineage markers (Ishii et al., 2011). Moreover, since all of the phenotypic markers for LT-HSC were proposed and established by employing uncultured cells, one of the recent reports employing ex vivo expansion system combined with in vivo transplants demonstrated that a population of UCB CD133⁺/CD38^{neg} cells resembles the most primitive HSPCs (Ito et al., 2010). Thus, the CD133 antigen seems to be a very good marker for the most primitive HSPCs.

Recently, a population of VSELs was identified in murine BM that do not exhibit hematopoietic activity immediately after isolation, but later acquire full hematopoietic potential, including the ability to reconstitute long-term hematopoiesis in lethally irradiated recipients following co-culture/activation over OP9 stroma (Kucia et al., 2006b; Ratajczak et al., 2011). Based in part on this, it was proposed that in murine BM these cells fulfill the functional criteria for LT-HSCs (Ratajczak et al., 2011). Interestingly, these cells were also identified as precursors of MSCs indicative of multipotentiality (Taichman et al., 2010). A corresponding population of SSEA-4⁺/Oct-4⁺/CD133⁺/CD34⁺/CXCR4⁺/Lin^{neg}/CD45^{neg} cells was identified recently (Kucia et al., 2007; McGuckin et al., 2008; Zuba-Surma et al., 2010; Sovalat et al., 2011) in human UCB, BM, and mPB.

Because human VSELs are very rare, the processing of one UCB unit by a standard procedure employing multiparameter sorting requires up to 4 days. This is, of course, sub-optimal taking into consideration cell viability, the time of sorter usage, and the time commitment of a sorter operator.

Thus, a goal of the presently disclosed subject matter was to develop a faster large-scale isolation protocol. To speed up the procedure for VSEL sorting, the presently disclosed subject matter employs in some embodiments (i) hypotonic lysis removal of erythrocytes to obtain UCB nucleated cells; (ii) enrichment for CD133⁺ VSELs by immunomagnetic beads; and (iii) sorting of VSELs from erythrocyte-depleted/immunomagnetic paramagnetic bead-enriched CD133⁺ mononuclear cells by employing staining with an ALDEFLUOR® detection reagent combined with anti-CD133 (different epitope) and fluorochrome-conjugated anti-CD45 and anti-GlyA antibodies. By employing this strategy, four particular populations of cells, (i) CD45^{neg}/GlyA^{neg}/CD133⁺/ALDH^{high}; (ii) CD45^{neg}/GlyA^{neg}/CD133⁺/ALDH^{low}; (iii) CD45⁺/GlyA^{neg}/CD133⁺/ALDH^{high}; and (iv) CD45⁺/GlyA^{neg}/CD133⁺/ALDH^{low}; can be sorted and subsequently tested for hematopoietic activity.

It was observed that UCB-purified CD45^{neg}/GlyA^{neg}/CD133⁺/ALDH^{high} and CD45/GlyA^{neg}/CD133⁺/ALDH^{low} VSELs, like their murine counterparts, did not exhibit hematopoietic potential immediately after isolation. However, it was further observed that UCB-derived CD45^{neg} VSELs, like murine VSELs, human ES cells, or iPS cells, can become specified into the hematopoietic lineage in co-cultures over OP9 stromal cell feeder layers (Ji et al., 2008). This robust specification of human UCB-derived CD45^{neg} VSELs is supported by their ability to form hematopoietic colonies in vitro that express pan-hematopoietic CD45 antigen as well as several major lympho-hematopoietic lineage markers (CD41, GlyA, CD14, CD3, and CD19). While not wishing to be bound by any particular theory of operation, it is possible that the fact that human UCB-VSELs, like their murine BM-derived counterparts (Ratajczak at al., 2011), can be more efficiently expanded into the lympho-hematopoietic lineage than ES cells or iPS cells demonstrates that they are already more committed to hematopoiesis, and thus might correspond to a population of UCB-derived LT-HSCs. These small cells isolated from human UCB highly expressed Oct-4, Nanog, and SSEA-4 at both the mRNA and protein levels. Moreover, freshly isolated VSELs already expressed the HoxB-4 gene, which is not expressed in ES cells, but is ultimately required for their hematopoietic expansion (Daley, 2003; Abramovich et al., 2005).

Additionally, it was found that UCB-derived VSELs not only differentiated over OP9 stroma into clonogenic hematopoietic progenitors but also into HSCs, which are able to establish human-murine chimerism in immunodeficient NOD/SCID animals. The level of human-murine chimerism can depend on several factors, such as phenotype, the number of transplanted cells, or the severity of immunodeficiency in the mice employed as recipients (Ito et al., 2008). In the case of the presently disclosed subject matter, trypsinized OP9 co-cultures whose activity could be influenced by the presence of the infused OP9 cells were transplanted.

Thus, the data presented herein unequivocally demonstrated that the UCB-derived population of CD45^{neg} VSELs could differentiate into CD45⁺ HSPCs and that these extremely rare cells (˜10³/100 ml of UCB for CD45^{neg}/GlyA^{neg}/CD133⁺/ALDH^{low} cells and (˜4×10³/100 ml of UCB for CD45^{neg}/GlyA^{neg}/CD133⁺/ALDH^{high} cells) showed significant hematopoietic potential.

Taking into consideration the hematopoietic potential of UCB-derived VSELs, whether the number of these cells changes in BM in aplastic anemia patients could be tested. If VSELs could be protected from damage, they might offer an alternative source of autologous HSPCs for transplantation in aplastic patients. Furthermore, it has been demonstrated that murine VSELs are highly resistant to irradiation (Ratajczak et al., 2011), which suggests that human VSELs could also survive myeloablative conditioning therapy for transplantation and could stimulate hematopoiesis in the recipient after unsuccessful or partial engraftment of transplanted cells.

### REFERENCES

All references listed below, as well as all references cited in the instant disclosure including the Appendix, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
Abramovich et al. (2005) Ann N Y Acad Sci 1044:109-116.
Basiji et al. (2007) Clin Lab Med 27:653-670.
Bhatia et al. (1998) Nat Med 4:1038-1045.
Carninci et al. (1996) Genomics 37:327-336.
Crosby et al. (2001) Gastroenterology 120:534-544.
Daley (2003) Ann N Y Aced Sci 996:122-131.
Fiegel et al. (2003) Hepatology 37:148-154.
Gallacher et al. (2000) Blood 95:2813-2820.
Hess et al. (2004) Blood 104:1648-1655.
Hess et al. (2006) Blood 107:2162-2169.
Hess et al. (2008) Stem Cells 26:611-620.
Houssaint (1980) Cell Differ 9:269-279.
Ishii et al. (2011) Exp Hematology 39:203-213 e201.
Ito et al. (2008) Curr Top Microbiol Immunol 324:53-76.
Ito et al. (2010) Blood 115:257-260.
Ji et al. (2008) Stem Cells 26:2485-2495.
Julien et al. (2000) Biochemistry 39:75-85.
Jung et al. (1999) Science 284:1998-2003.
Kiel et al. (2005) Cell 121:1109-1121.
Kimura et al. (2007) Stem Cells 25:1348-1355.
Krupnick et al. (2004) Tissue Eng 10:723-735.
Kucia et al. (2006b) Leukemia 20:857-869.
Kucia et al. (2007) Leukemia 21:297-303.
Larochelle et al. (1996) Nat Med 2:1329-1337.
Lemmer et al. (1998) J Hepatol 29:450-454.
McGuckin et al. (2008) Nat Protoc 3:1046-1055.
Minguet et al. (2003) J Clin Invest 112:1152-1163.
Nava et al. (2005) Differentiation 73:249-260.
Nierhoff et al. (2005) Hepatology 42:130-139.
Nowak et al. (2005) Gut 54:972-979.
PCT International Patent Application Publication No. WO 2007/067280.
PCT International Patent Application Publication No. 2009/059032.
Peterson et al. (1998) Hepatology 27:433-445.
Ratajczak (2008) Curr Opin Hematol 15:293-300.
Ratajczak et al. (2003) Stem Cells 21:363-371.
Ratajczak et al. (2004) Leukemia 18:29-40.
Ratajczak et al. (2007) Leukemia 21:860-867.
Ratajczak et al. (2008a) Exp Hematol 36:742-751.
Ratajczak et al. (2008b) J Autoimmun 30:151-162.
Ratajczak et al. (2008c) Stem Cell Rev 4:89-99.
Ratajczak et al. (2011) Exp Hematology 39:225-237,
Rossi et al, (2001) Genes Dev 15:1998-2009.
Shin et al. (2008) Blood 112:385.
Sovalat et al. (2011) Exp Hematology 39:495-505.
Suzuki & Nakauchi (2002) Semin Cell Dev Biol 13:455-461.
Suzuki et al. (2000) Hepatology 32:1230-1239.
Suzuki et al. (2002) J Cell Biol 156:173-184.
Tada et al. (2006) Anat Histol Embryol 35:235-240.
Taichman et al. (2910) Stem Cells Dev 19:1557-1570.
Tang & Casey (1999) Biochemistry 38:14565-14572.
Tavian & Peault (2005) Int J Dev Biol 49:243-250.
U.S. Patent Application Publication No. 2009/015522.
U.S. Parent Application Publication No. 2010/0267107.
U.S. Pat. No. 5,168,037.
Wang et al. (2003) Blood 101:2924-2931.
Zaret (2000) Mach Dev 92:83-88.
Zaret (2001) Curr Opin Genet Dev 11:568-574.
Zaret (2002) Nat Rev Genet 3:499-512.
Zuba-Surma et al. (2007a) Folia Histochem Cytobiol 45:279-290,
Zuba-Surma et al. (2010) Eur J Haematol 84:34-46.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for purifying a very small embryonic-like (VSEL) stem cell from a population of cells suspected of comprising VSEL stem cells, the method comprising:
(a) providing a population of cells suspected of comprising a VSEL stem cell; and
(b) isolating a CD45^{neg}/GlyA^{neg}/CD133⁺/ALDH^{high} subpopulation, a CD45^{neg}/GlyA^{neg}/CD133⁺/ALDH^{low} subpopulation, a CD45^{neg}/Lin^{neg}/SSEA-4⁺/ALDH^{high} subpopulation, a CD45^{neg}/Lin^{neg}/SSEA-4/ALDH^{low} subpopulation, or any combination thereof from the population,
whereby a VSEL stem cell is purified from the population.

2. The method of claim 1, wherein the isolating step comprises employing anti-CD133 paramagnetic beads to isolate a CD133⁺ subpopulation from the population and/or comprises employing an anti-SSEA-4 antibody to isolate an SSEA-4⁺ subpopulation from the population, or both.

3. The method of claim 1, wherein the isolating step comprises employing a fluorescent dye to detect aldehyde dehydrogenase (ALDH) expression in the cells of the population, the CD133⁺ subpopulation from the population, the SSEA-4⁺ subpopulation from the population, or in any other subpopulation thereof.

4. The method of claim 1, wherein the fluorescent dye is employed for separating the cells into ALDH^{high} and ALDH^{low} fractions.

5. The method of claim 1, wherein the isolating comprises employing a reagent that binds to Glycophorin A (GlyA) to remove GlyA⁺ cells from the population.

6. The method of claim 1, wherein the isolating comprises employing a reagent that binds to CD45 to remove CD45⁺ cells from the population.

7. The method of claim 1, wherein the population of cells suspected of comprising VSEL stem cells is a bone marrow sample, a peripheral blood sample, a spleen sample, an umbilical cord blood sample, or any combination thereof.

8. A method for generating an in vitro hematopoietic colony derived from a very small embryonic-like (VSEL) stem cell, the method comprising:
(a) providing a CD45^{neg}/GlyA^{neg}/CD133⁺/ALDH^{high} subpopulation, a CD45^{neg}/GlyA^{neg}/CD133⁺/ALDH^{high} subpopulation, a CD45^{neg}/Lin^{neg}/SSEA-4⁺/ALDH^{high} subpopulation, a CD45^{neg}/Lin^{neg}/SSEA-4/ALDH^{low} subpopulation, or any combination thereof purified by the method of claim 1; and
(b) co-culturing the VSEL stem cell present therein in the presence of an OP9 stromal cell feeder layer under conditions sufficient to generate an in vitro hematopoietic colony derived from the VSEL stem cell.

9. The method of claim 8, wherein the conditions sufficient to generate an in vitro hematopoietic colony derived from the VSEL stem cell comprise co-culturing the VSEL stem cell in the presence of the OP9 stromal cell feeder layer for at least 5 days, optionally for at least 7 days, and further optionally for at least 10 days.

10. A method for generating lympho-hematopoietic chimerism in a subject, the method comprising introducing into a lympho-hematopoietic compartment of the subject a plurality of CD45^{neg}/GlyA^{neg}/CD133⁺/ALDH^{high} cells comprising VSEL stem cells, a plurality of CD45^{neg}/GlyA^{neg}/CD133⁺/ALDH^{low} cells comprising VSEL stem cells, a plurality of CD45^{neg}/Lin^{neg}/SSEA-4⁺/ALDH^{high} cells comprising VSEL stem cells, a plurality of CD45^{neg}/Lin^{neg}/SSEA-4⁺/ALDH^{low} cells comprising VSEL stem cells, or a combination thereof, wherein the plurality of CD45^{neg}/GlyA^{neg}/CD133⁺/ALDH^{high} cells comprising VSEL stem cells, the plurality of CD45^{neg}/GlyA^{neg}/CD133⁺/ALDH^{low} cells comprising VSEL stem cells, the plurality of CD45^{neg}/Lin^{neg}/SSEA-4⁺/ALDH^{high} cells comprising VSEL stem cells, the plurality of CD45^{neg}/Lin^{neg}/SSEA-4⁺/ALDH^{low} cells comprising VSEL stem cells, or the combination thereof were cultured over an OP9 stromal cell feeder layer under conditions sufficient to induce lympho-hematopoietic competency in one or more VSEL stem cells present therein.

11. The method of claim 10, wherein the introducing comprises administering the plurality of CD45^{neg}/GlyA^{neg}/CD133⁺/ALDH^{high} cells comprising VSEL stem cells, the plurality of CD45^{neg}/GlyA^{neg}/CD133⁺/ALDH^{low} cells comprising VSEL stem cells, the plurality of CD45^{neg}/Lin^{neg}/SSEA-4⁺/ALDH^{high} cells comprising VSEL stem cells, the plurality of CD45^{neg}/Lin^{neg}/SSEA-4⁺/ALDH^{low} cells comprising VSEL stem cells, or the combination thereof to the subject intravenously.

12. The method of claim 11, wherein the introducing step comprises a sufficient number of VSEL stem cells to repopulate bone marrow of the subject with lympho-hematopoietic cells derived from the VSEL stem cells.

13. The method of claim 10, wherein the subject is a mammal, optionally a human.
